(19) United States
(12) Patent Application Publication
(10) Pub. No.: US 2005/0101548 A1
(43) Pub. Date: May 12, 2005

(54) Antimicrobial derivatives

(76) Inventors: Xiaodong Lin, Orinda, CA (US); Daniel Chu, Santa Clara, CA (US)

Correspondence Address: Chiron Corporation;Intellectual Property, P.O. Box 8097, Emeryville, CA, 94662-8097, US

(21) Appl. No.: 10/940,900

(22) Filed: Sep. 13, 2004

Related U.S. Application Data
(60) Provisional application No. 60/502,419, filed on Sep. 12, 2003.

(51) Int. Cl. C07H 17/08; A61K 31/7048

(52) U.S. Cl. 514/28; 536/7.4

(57) ABSTRACT

Antimicrobial compounds are provided having the formula (Q): 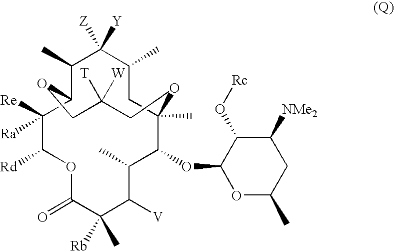 as well as tautomers, stereoisomers, pharmaceutically acceptable salts, esters or prodrugs thereof; pharmaceutical compositions comprising such compounds; methods of treating bacterial infections by the administration of such compounds; and processes for the preparation of the compounds.

### CROSS-REFERENCE TO RELATED APPLICATION

This application claims the benefit of U.S. patent application No. 60/502,419, filed Sep. 12, 2003. The disclosure of the above provisional application is herein incorporated by reference in its entirety and for all purposes as if fully set forth herein.

### BACKGROUND OF THE INVENTION

Erythromycins A through D, represented by formula (I),

| | |
|---|---|
| | (I) |
| 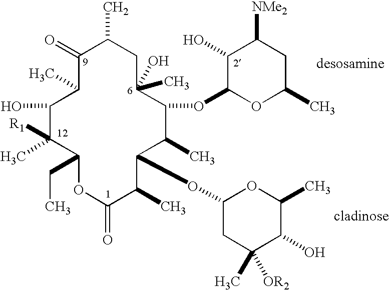 | |

| Erythromycin | R1 | R2 |
|---|---|---|
| A | —OH | —CH₃ |
| B | —H | —CH₃ |
| C | —OH | —H |
| D | —H | —H |

are well-known and potent antibacterial agents, and are used widely to treat and prevent bacterial infections. As with other antibacterial agents however, bacterial strains having resistance or insufficient susceptibility to erythromycin have been identified. Also, erythromycin A has only weak activity against Gram-negative bacteria. Therefore, there is a continuing need to identify new erythromycin derivative compounds which possess improved antibacterial activity, which have less potential for developing resistance, which possess Gram-negative activity, or which possess unexpected selectivity against target microorganisms. Consequently, numerous investigators have prepared chemical derivatives of erythromycin in an attempt to obtain analogs having modified or improved profiles of antibiotic activity. For example, the compound 6-OMe erythromycin A, or clarithromycin, has found widespread use. However, even this compound is beginning to lose its effectiveness and other erythromycin derivatives having improved activity are needed. Other 6-O-substituted erythromycin compounds have also been proposed for this purpose. For example, PCT application WO 92/09614, published Jun. 11, 1992, discloses tricyclic 6-O-methylerythromycin A derivatives. U.S. Pat. No. 5,444,051 discloses 6-O-substituted-3-oxoerythromycin A derivatives in which the substituents are selected from alkyl, —CONH₂, —CONHC(O)alkyl and —CONHSO₂ alkyl. PCT application WO 97/10251, published Mar. 20, 1997, discloses 6-O-methyl 3-descladinose erythromycin derivatives. European Patent Application 596802, published May 11, 1994, discloses bicyclic 6-O-methyl-3-oxoerythromycin A derivatives.

A class of 3-O ketolide erythromycin derivatives has been disclosed in U.S. Pat. Nos. 6,147,197 and 5,635,485. Representative lead compounds in this class include, for example ABT-773 disclosed in U.S. Pat. No. 6,147,197 and telithromycin disclosed in U.S. Pat. No. 5,635,485. The structures of these compounds are as follows:

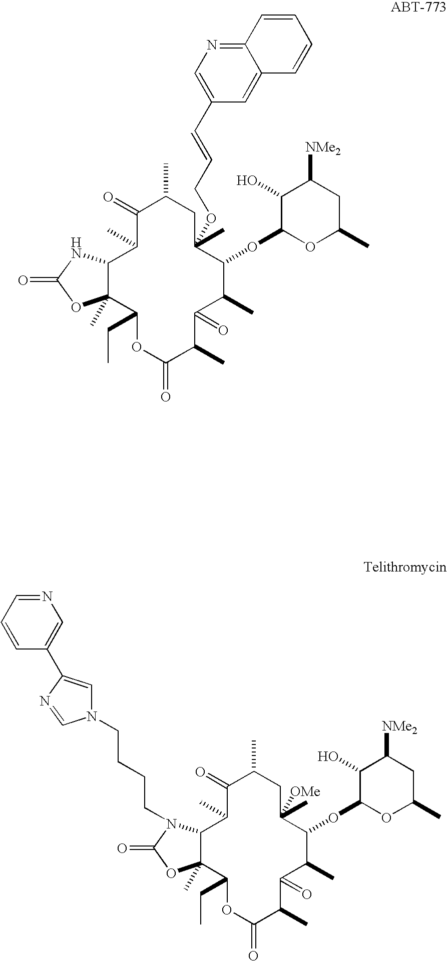

Other ketolide modifications include, for example, those shown in U.S. Pat. No. 6,124,269 and International Application Publication No. WO 00/69875. Macrolides and ketolides containing non-methyl C12 groups are disclosed in WO 03/004509, the disclosures of which are incorporated herein by reference.

Recently, a class of bridged ketolides have been described (Enanta Pharmaceuticals, 8^{th} International Antibacterial Drug Discovery & Development Summit, Strategic Research Institute®, Mar. 24-25, 2003, Princeton N.J.). Representative bridged ketolide compounds include, for example, EP-1304, which corresponds to the following structure: 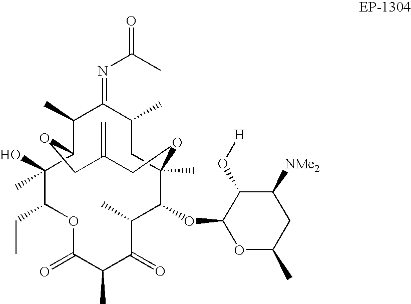
EP-1304 exhibits activity against some macrolide-sensitive and resistant bacteria in vivo. Analogs of EP-1304, such as EP-1562 and EP-12728, have been shown to possess antibacterial activity against S. pneumoniae. The structure of compounds EP-1562 and EP-12728 are as follows:

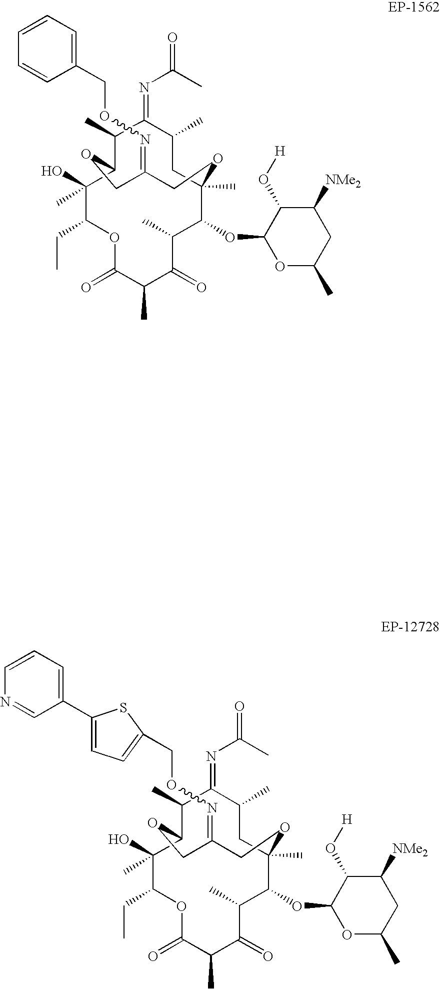

Other C6-C11 bridged ketolide compounds are disclosed in WO 03/097659, WO 04/011009, and WO 04/011477, the disclosures of which are incorporated herein by reference.

There exists a continuing medical need to identify new macrolide and ketolide derivatives that possess improved antibacterial activity, less potential for developing resistance, activity against Gram-negative bacteria, increased selectivity against target microorganisms, as well as a better safety profile. In an effort to address that need, the inventors herein present chemical derivatives of ketolides to obtain analogs having modified and/or improved pharmacokinetic profiles of antibiotic activity over ketolide compounds known in the art.

### SUMMARY OF THE INVENTION

The present invention provides novel bridged macrolide and ketolide derivatives. The present invention also provides useful common intermediates, methods for their synthesis, and methods of use of such compounds for the treatment and/or prophylaxis of diseases, especially bacterial infections.

In one embodiment, the present invention provides compounds of the following formula (Q): 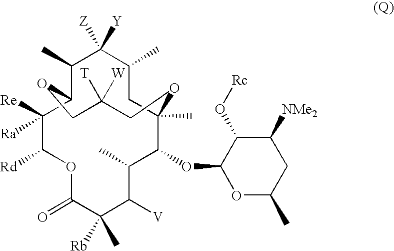
or a stereoisomer, tautomer, pharmaceutically acceptable salt, ester or prodrug thereof, wherein
V is —OCORx, carbonyl, or a cladinose moiety of the formula: 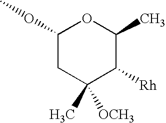
   wherein Rx is H, alkyl, —O-alkyl, —N(H)-alkyl, or —N(alkyl)₂;
either Y and Z taken together define a group X, wherein X is selected from the group consisting of
(1) ═O,
(2) ═N—OH,
(3) ═N—O—R¹, wherein R¹ is selected from the group consisting of
   (a) C₁-C₁₂-alkyl,
   (b) C₁-C₁₂-alkyl substituted with alkoxy,
   (c) C₁-C₁₂-alkyl substituted with aryl,
   (d) C₁-C₁₂-alkyl substituted with substituted aryl,
   (e) C₁-C₁₂-alkyl substituted with heteroaryl,
   (f) C₁-C₁₂-alkyl substituted with substituted heteroaryl,
   (g) C₃-C₁₂-cycloalkyl, and
   (h) —Si—(R²)(R³)(R⁴), wherein R², R³, and R⁴ are each independently selected from C₁-C₁₂-alkyl and aryl;
(4) ═N—O—C(R⁵)(R⁶)—O—R¹, wherein R¹ is as previously defined and R⁵ and R⁶ are each independently selected from the group consisting of
   (a) hydrogen,
   (b) C₁-C₁₂-alkyl,
   (c) C₁-C₁₂-alkyl substituted with aryl,
   (d) C₁-C₁₂-alkyl substituted with substituted aryl,
   (e) C₁-C₁₂-alkyl substituted with heteroaryl, and
   (f) C₁-C₁₂-alkyl substituted with substituted heteroaryl,
or R⁵ and R⁶ taken together with the atoms to which they are attached form a C₃-C₁₂-cycloalkyl ring;
(5) ═N—C(O)R¹⁵, wherein R¹⁵ is selected from the group consisting of
   (a) C₁-C₁₂-alkyl,
   (b) substituted C₁-C₁₂-alkyl,
   (c) C₂-C₁₂-alkenyl,
   (d) substituted C₂-C₁₂-alkenyl,
   (e) C₂-C₁₂-alkynyl,
   (f) substituted C₂-C₁₂-alkynyl,
   (g) aryl,
   (h) C₃-C₈-cycloalkyl,
   (i) substituted C₃-C₈-cycloalkyl,
   (j) substituted aryl,
   (k) heterocycloalkyl,
   (l) substituted heterocycloalkyl,
   (m) C₁-C₁₂-alkyl substituted with aryl,
   (n) C₁-C₁₂-alkyl substituted with substituted aryl,
   (o) C₁-C₁₂-alkyl substituted with heterocycloalkyl,
   (p) C₁-C₁₂-alkyl substituted with substituted heterocycloalkyl,
   (q) C₁-C₁₂-alkyl substituted with C₃-C₈-cycloalkyl,
   (r) C₁-C₁₂-alkyl substituted with substituted C₃-C₈-cycloalkyl,
   (s) heteroaryl,
   (t) substituted heteroaryl,
   (u) C₁-C₁₂-alkyl substituted with heteroaryl, and
   (v) C₁-C₁₂-alkyl substituted with substituted heteroaryl;
   (w) C₁-C₁₂-alkyl substituted with alkylalkoxy,
   (x) C₁-C₆-alkoxy,
   (y) C₁-C₆-thioalkoxy,
   (z) amino,
   (aa) alkylamino,
   (bb) dialkylamino,
   (cc) —NO₂, and
   (dd) —COOH,
      or one of Y and Z is hydrogen and the other is selected from a group consisting of
(1) hydroxyl,
(2) protected hydroxyl, and
(3) —NR⁷R⁸, wherein R⁷ and R⁸ are independently selected from hydrogen and alkyl, substituted alkyl, or R⁷ and R⁸ are taken with the nitrogen atom to which they are connected to form a 3- to 7-membered ring which, when the ring is a 5- to 7-membered ring, may optionally contain a hetero function selected from the group consisting of —O—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or —S(O)ₙ—, wherein n is 1 or 2;
Ra is selected from the group consisting of
(1) hydrogen;
(2) C₁ alkyl further substituted with one or more substituents selected from a group consisting of
   (a) hydroxyl,
   (b) halogen,
   (c) thiol, which can be further substituted with an C₁-C₁₂-alkyl or substituted C₁-C₁₂-alkyl group,
   (d) C₁-C₁₂-alkyl, which can be further substituted by halogen, hydroxyl, C₁-C₁₂-alkoxy, or amino,
   (e) C₁-C₃-alkoxy,
   (f) C₁-C₃-thioalkoxy,
   (g) amino,
   (h) C₁-C₁₂-alkylamino,
   (i) C₁-C₁₂-dialkylamino,
   (j) —CN,
   (k) —NO₂,
   (l) —CONH₂,
   (m) —COOH,
   (n) —CO₂R¹⁰, wherein R¹⁰ is C₁-C₃-alkyl, aryl substituted with C₁-C₃-alkyl, or heteroaryl substituted with C₁-C₃-alkyl,
   (o) —N₃;
(3) C₂-C₁₂-alkyl;
(4) substituted C₁-C₁₂-alkyl;
(5) C₂-C₄-alkenyl, which can be further substituted with C₁-C₁₂-alkyl and one or more halogen groups;
(6) C₂-C₄-alkynyl, which can be further substituted with C₁-C₁₂-alkyl and one or more halogen groups;
(7) aryl, which can be further substituted with C₁-C₁₂-alkyl and one or more halogen groups;
(8) —CHO;
(9) —CO₂H;
(10) —CN;
(11) —CO₂R¹⁰, wherein R¹⁰ is as previously defined;
(12) —C(O)NR¹¹R¹², wherein R¹¹ and R¹² are independently selected from hydrogen, C₁-C₃-alkyl, C₁-C₃-alkyl substituted with aryl, substituted aryl, heteroaryl, and substituted heteroaryl;
(13) —C(O)R⁹, wherein R⁹ is selected from the group consisting of
   (a) alkyl optionally substituted with a substituent selected from the group consisting of
      (i) aryl,
      (ii) substituted aryl,
      (iii) heteroaryl, and
      (iv) substituted heteroaryl,
   (b) aryl,
   (c) substituted aryl,
   (d) heteroaryl,
   (e) substituted heteroaryl, and
   (f) heterocycloalkyl, and
(14) thioester;
Rb is hydrogen, halogen, or C₁-C₁₂-alkyl which can be further substituted by one or more halo groups, or Rb can be taken together with V to form a double bond;
Rc is hydrogen or a hydroxyl protecting group;
Rd is selected from the group consisting of
(1) C₁-C₁₂-alkyl,
(2) C₁-C₁₂-alkyl substituted with one or more substituents selected from the group consisting of
   (a) halogen,
   (b) hydroxyl, and
   (c) C₁-C₃-alkoxy,
(3) C₃-C₇-cycloalkyl,
(4) C₂-C₄-alkenyl, and
(5) C₂-C₄-alkynyl;
Re is hydroxyl, amino, or alkylamino; or Re and Ra may be taken together to form an epoxide, a carbonyl, an olefin, or a substituted olefin; or Re and Ra when taken together with the atom to which they are attached form a spiro ring consisting of C₃-C₇-carbocyclic, carbonate, or carbamate, wherein the nitrogen atom can be unsubstituted or substituted with an alkyl group;
Rh is selected from the group consisting of
(1) hydrogen,
(2) —ORj, wherein Rj is hydrogen or a hydroxyl protecting group,
(3) halogen, and
(4) —OC(O)NHRi, wherein Ri is selected from a group consisting of
   (a) C₁-C₄ alkyl,
   (b) C₁-C₄ aminoalkyl where the amino group is substituted with one or two groups selected from
      (i) C₁-C₄ alkyl,
      (ii) C₁-C₄ alkyl substituted with halogen,
      (iii) C₁-C₄ alkyl substituted with alkoxy,
      (iv) C₁-C₄ alkyl substituted with hydroxyl,
      (v) C₁-C₄ alkyl substituted with aryl,
      (vi) C₁-C₄ alkyl substituted with substituted aryl,
      (vii) C₁-C₄ alkyl substituted with heteroaryl,
      (viii) C₁-C₄ alkyl substituted with substituted heteroaryl, and
      (ix) C₃-C₆ cycloalkyl;
T and W taken together define a group R, wherein R is selected from the group consisting of
(1) ═O;
(2) ═CH(Rk), wherein Rk is a cis or trans substituent selected from the group consisting of
   (a) hydrogen,
   (b) halogen selected from the group consisting of Br, Cl, F, and I,
   (c) C₁-C₁₂-alkyl,
   (d) C₁-C₁₂-alkyl substituted with aryl,
   (e) C₁-C₁₂-alkyl substituted with substituted aryl,
   (f) C₁-C₁₂-alkyl substituted with heteroaryl,
   (g) C₁-C₁₂-alkyl substituted with substituted heteroaryl,
(h) C₂-C₁₂-alkenyl,
   (i) C₂-C₁₂-alkenyl substituted with aryl,
   (j) C₂-C₁₂-alkenyl substituted with substituted aryl,
   (k) C₂-C₁₂-alkenyl substituted with heteroaryl,
   (l) C₂-C₁₂-alkenyl substituted with substituted heteroaryl,
   (m) C₂-C₁₂-alkynyl,
   (n) C₂-C₁₂-alkynyl substituted with aryl,
   (o) C₂-C₁₂-alkynyl substituted with substituted aryl,
   (p) C₂-C₁₂-alkynyl substituted with heteroaryl,
   (q) C₂-C₁₂-alkynyl substituted with substituted heteroaryl,
   (r) aryl,
   (s) substituted aryl,
   (t) heteroaryl,
   (u) substituted heteroaryl; and
(3) ═N—O-(Rm), wherein Rm is selected from the group consisting of
   (a) hydrogen,
   (b) C₁-C₁₂-alkyl,
   (c) C₁-C₁₂-alkyl substituted with C₁-C₁₂-alkenyl,
   (d) C₁-C₁₂-alkyl substituted with substituted C₁-C₁₂-alkenyl,
   (e) C₁-C₁₂-alkyl substituted with aryl,
   (f) C₁-C₁₂-alkyl substituted with substituted aryl,
   (g) C₁-C₁₂-alkyl substituted with heteroaryl,
   (h) C₁-C₁₂-alkyl substituted with substituted heteroaryl,
   (i) C₂-C₁₂-alkenyl,
   (j) C₂-C₁₂-alkenyl substituted with aryl,
   (k) C₂-C₁₂-alkenyl substituted with substituted aryl,
   (l) C₂-C₁₂-alkenyl substituted with heteroaryl,
   (m) C₂-C₁₂-alkenyl substituted with substituted heteroaryl,
   (n) aryl,
   (o) substituted aryl,
   (p) heteroaryl, and
   (q) substituted heteroaryl.

The present invention also provides pharmaceutical compositions which comprise a therapeutically effective amount of a compound as defined above in combination with a pharmaceutically acceptable carrier.

The invention further relates to methods of treating bacterial infections in a host mammal in need of such treatment comprising administering to a mammal in need of such treatment a therapeutically effective amount of a compound of the invention as defined above. Preferred mammals are humans and domesticated animals such as dogs, cats, horses, cows, cattle, sheep, pigs, goats, lamas, donkeys, and mice. An especially preferred mammal is a human.

### DETAILED DESCRIPTION OF THE PREFERRED EMBODIMENT ABBREVIATIONS

Abbreviations which have been used in the descriptions of the scheme and the examples that follow are: Ac for acetyl; AcOH for acetic acid; AIBN for azobisisobutyronitrile; Bu₃SnH for tributyltin hydride; CDI for carbonyldiimidazole; DBU for 1,8-diazabicyclo[5.4.0]undec-7-ene; DCM for dichloromethane; DEAD for diethylazodicarboxylate; DMF for dimethylformamide; DMP for 2,2-dimethoxypropane DMSO for dimethylsulfoxide; DPPA for diphenylphosphoryl azide; Et for ethyl; Et₃N for triethylamine; EtOAc for ethyl acetate; Et₂O for diethyl ether; EtOH for ethanol; HOAc for acetic acid; LiHMDS or LiN(TMS)₂ for lithium bis(trimethylsilyl)amide; MCPBA for meta-chloroperbenzoic acid; Me for methyl; MeOH for methanol; MsCl for methanesulfonyl chloride; NaHMDS or NaN(TMS)₂ for sodium bis(trimethylsilyl)amide; NMO for N-methylmorpholine N-oxide; SOCl₂ for thionyl chloride; PPTS for pyridium p-toluene sulfonate; Py for pyridine; TEA for triethylamine; THF for tetrahydrofuran; TMSCl for trimethylsilyl chloride; TMSCF₃ for trimethyl(trifluoromethyl)-silane; TPP for triphenylphosphine; TPAP for tetra-n-propylammonium perruthenate; DMAP for 4-dimethylamino pyridine, TsOH for p-toluene sulfonic acid.

In one embodiment, the present invention provides compounds of the following formula (Q): 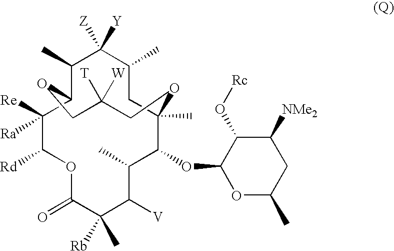
or a stereoisomer, tautomer, pharmaceutically acceptable salt, ester or prodrug thereof, wherein
V is —OCORx, carbonyl, or a cladinose moiety of the formula: 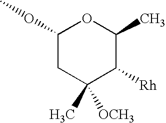
   wherein Rx is H, alkyl, —O-alkyl, —N(H)-alkyl, or —N(alkyl)₂;
either Y and Z taken together define a group X, wherein X is selected from the group consisting of
(1) ═O,
(2) ═N—OH,
(3) ═N—O—R¹, wherein R¹ is selected from the group consisting of
   (a) C₁-C₁₂-alkyl,
   (b) C₁-C₁₂-alkyl substituted with alkoxy,
   (c) C₁-C₁₂-alkyl substituted with aryl,
   (d) C₁-C₁₂-alkyl substituted with substituted aryl,
   (e) C₁-C₁₂-alkyl substituted with heteroaryl,
   (f) C₁-C₁₂-alkyl substituted with substituted heteroaryl,
   (g) C₃-C₁₂-cycloalkyl, and
   (h) —Si—(R²)(R³)(R⁴), wherein R², R³, and R⁴ are each independently selected from C₁-C₁₂-alkyl and aryl;
(4) ═N—O—C(R⁵)(R⁶)—O—R′, wherein R′ is as previously defined and R⁵ and R⁶ are each independently selected from the group consisting of
   (a) hydrogen,
   (b) C₁-C₁₂-alkyl,
   (c) C₁-C₁₂-alkyl substituted with aryl,
   (d) C₁-C₁₂-alkyl substituted with substituted aryl,
   (e) C₁-C₁₂-alkyl substituted with heteroaryl, and
   (f) C₁-C₁₂-alkyl substituted with substituted heteroaryl,
or R⁵ and R⁶ taken together with the atoms to which they are attached form a C₃-C₁₂-cycloalkyl ring;
(5) ═N—C(O)R¹⁵, wherein R¹⁵ is selected from the group consisting of
   (a) C₁-C₁₂-alkyl,
   (b) substituted C₁-C₁₂-alkyl,
   (c) C₂-C₁₂-alkenyl,
   (d) substituted C₂-C₁₂-alkenyl,
   (e) C₂-C₁₂-alkynyl,
   (f) substituted C₂-C₁₂-alkynyl,
   (g) aryl,
   (h) C₃-C₈-cycloalkyl,
   (i) substituted C₃-C₈-cycloalkyl,
   (j) substituted aryl,
   (k) heterocycloalkyl,
   (l) substituted heterocycloalkyl,
   (m) C₁-C₁₂-alkyl substituted with aryl,
   (n) C₁-C₁₂-alkyl substituted with substituted aryl,
   (o) C₁-C₁₂-alkyl substituted with heterocycloalkyl,
   (p) C₁-C₁₂-alkyl substituted with substituted heterocycloalkyl,
   (q) C₁-C₁₂-alkyl substituted with C₃-C₈-cycloalkyl,
   (r) C₁-C₁₂-alkyl substituted with substituted C₃-C₈-cycloalkyl,
   (s) heteroaryl,
   (t) substituted heteroaryl,
   (u) C₁-C₁₂-alkyl substituted with heteroaryl, and
   (v) C₁-C₁₂-alkyl substituted with substituted heteroaryl;
   (w) C₁-C₁₂-alkyl substituted with alkylalkoxy,
   (x) C₁-C₆-alkoxy,
   (y) C₁-C₆-thioalkoxy,
   (z) amino,
   (aa) alkylamino,
   (bb) dialkylamino,
   (cc) —NO₂, and
   (dd) —COOH,
      or one of Y and Z is hydrogen and the other is selected from a group consisting of
(1) hydroxyl,
(2) protected hydroxyl, and
(3) —NR⁷R⁸, wherein R⁷ and R⁸ are independently selected from hydrogen and alkyl, substituted alkyl, or R⁷ and R⁸ are taken with the nitrogen atom to which they are connected to form a 3- to 7-membered ring which, when the ring is a 5- to 7-membered ring, may optionally contain a hetero function selected from the group consisting of —O—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or —S(O)ₙ—, wherein n is 1 or 2;
Ra is selected from the group consisting of
(1) hydrogen;
(2) C₁ alkyl further substituted with one or more substituents selected from a group consisting of
   (a) hydroxyl,
   (b) halogen,
   (c) thiol, which can be further substituted with an C₁-C₁₂-alkyl or substituted C₁-C₁₂-alkyl group,
   (d) C₁-C₁₂-alkyl, which can be further substituted by halogen, hydroxyl, C₁-C₁₂-alkoxy, or amino,
   (e) C₁-C₃-alkoxy,
   (f) C₁-C₃-thioalkoxy,
   (g) amino,
   (h) C₁-C₁₂-alkylamino,
   (i) C₁-C₁₂-dialkylamino,
   (j) —CN,
   (k) —NO₂,
   (l) —CONH₂,
   (m) —COOH,
   (n) —CO₂R¹⁰, wherein R¹⁰ is C₁-C₃-alkyl, aryl substituted with C₁-C₃-alkyl, or heteroaryl substituted with C₁-C₃-alkyl,
   (o) —N₃;
(3) C₂-C₁₂-alkyl;
(4) substituted C₁-C₁₂-alkyl;
(5) C₂-C₄-alkenyl, which can be further substituted with C₁-C₁₂-alkyl and one or more halogen groups;
(6) C₂-C₄-alkynyl, which can be further substituted with C₁-C₁₂-alkyl and one or more halogen groups;
(7) aryl, which can be further substituted with C₁-C₁₂-alkyl and one or more halogen groups;
(8) —CHO;
(9) —CO₂H;
(10) —CN;
(11) —CO₂R¹⁰, wherein R¹⁰ is as previously defined;
(12) —C(O)NR¹¹R¹², wherein R¹¹ and R¹² are independently selected from hydrogen, C₁-C₃-alkyl, C₁-C₃-alkyl substituted with aryl, substituted aryl, heteroaryl, and substituted heteroaryl;
(13) —C(O)R⁹, wherein R⁹ is selected from the group consisting of
   (a) alkyl optionally substituted with a substituent selected from the group consisting of
      (i) aryl,
      (ii) substituted aryl,
      (iii)heteroaryl, and
      (iv) substituted heteroaryl,
   (b) aryl,
   (c) substituted aryl,
   (d) heteroaryl,
   (e) substituted heteroaryl, and
   (f) heterocycloalkyl, and
(14) thioester;
Rb is hydrogen, halogen, or C₁-C₁₂-alkyl which can be further substituted by one or more halo groups, or Rb can be taken together with V to form a double bond;
Rc is hydrogen or a hydroxyl protecting group;
Rd is selected from the group consisting of
(1) C₁-C₁₂-alkyl,
(2) C₁-C₁₂-alkyl substituted with one or more substituents selected from the group consisting of
   (a) halogen,
   (b) hydroxyl, and
   (c) C₁-C₃-alkoxy,
(3) C₃-C₇-cycloalkyl,
(4) C₂-C₄-alkenyl, and
(5) C₂-C₄-alkynyl;
Re is hydroxyl, amino, or alkylamino; or Re and Ra may be taken together to form an epoxide, a carbonyl, an olefin, or a substituted olefin; or Re and Ra when taken together with the atom to which they are attached form a spiro ring consisting of C₃-C₇-carbocyclic, carbonate, or carbamate, wherein the nitrogen atom can be unsubstituted or substituted with an alkyl group;
Rh is selected from the group consisting of
(1) hydrogen,
(2) —ORj, wherein Rj is hydrogen or a hydroxyl protecting group,
(3) halogen, and
(4) —OC(O)NHRi, wherein Ri is selected from a group consisting of
   (a) C₁-C₄ alkyl,
   (b) C₁-C₄ aminoalkyl where the amino group is substituted with one or two groups selected from
      (i) C₁-C₄ alkyl,
      (ii) C₁-C₄ alkyl substituted with halogen,
      (iii) C₁-C₄ alkyl substituted with alkoxy,
      (iv) C₁-C₄ alkyl substituted with hydroxyl,
      (v) C₁-C₄ alkyl substituted with aryl,
      (vi) C₁-C₄ alkyl substituted with substituted aryl,
      (vii) C₁-C₄ alkyl substituted with heteroaryl,
      (viii) C₁-C₄ alkyl substituted with substituted heteroaryl, and
      (ix) C₃-C₆ cycloalkyl;
T and W taken together define a group R, wherein R is selected from the group consisting of
(1) ═O;
(2) ═CH(Rk), wherein Rk is a cis or trans substituent selected from the group consisting of
   (a) hydrogen,
   (b) halogen selected from the group consisting of Br, Cl, F, and I,
   (c) C₁-C₁₂-alkyl,
   (d) C₁-C₁₂-alkyl substituted with aryl,
   (e) C₁-C₁₂-alkyl substituted with substituted aryl,
   (f) C₁-C₁₂-alkyl substituted with heteroaryl,
   (g) C₁-C₁₂-alkyl substituted with substituted heteroaryl,
   (h) C₂-C₁₂-alkenyl,
   (i) C₂-C₁₂-alkenyl substituted with aryl,
   (j) C₂-C₁₂-alkenyl substituted with substituted aryl,
   (k) C₂-C₁₂-alkenyl substituted with heteroaryl,
   (l) C₂-C₁₂-alkenyl substituted with substituted heteroaryl,
   (m) C₂-C₁₂-alkynyl,
   (n) C₂-C₁₂-alkynyl substituted with aryl,
   (o) C₂-C₁₂-alkynyl substituted with substituted aryl,
   (p) C₂-C₁₂-alkynyl substituted with heteroaryl,
   (q) C₂-C₁₂-alkynyl substituted with substituted heteroaryl,
   (r) aryl,
   (s) substituted aryl,
   (t) heteroaryl,
   (u) substituted heteroaryl; and
(3) ═N—O-(Rm), wherein Rm is selected from the group consisting of
   (a) hydrogen,
   (b) C₁-C₁₂-alkyl,
   (c) C₁-C₁₂-alkyl substituted with C₁-C₁₂-alkenyl,
   (d) C₁-C₁₂-alkyl substituted with substituted C₁-C₁₂-alkenyl,
   (e) C₁-C₁₂-alkyl substituted with aryl,
   (f) C₁-C₁₂-alkyl substituted with substituted aryl,
   (g) C₁-C₁₂-alkyl substituted with heteroaryl,
   (h) C₁-C₁₂-alkyl substituted with substituted heteroaryl,
   (i) C₂-C₁₂-alkenyl,
   (j) C₂-C₁₂-alkenyl substituted with aryl,
   (k) C₂-C₁₂-alkenyl substituted with substituted aryl,
   (l) C₂-C₁₂-alkenyl substituted with heteroaryl,
   (m) C₂-C₁₂-alkenyl substituted with substituted heteroaryl,
   (n) aryl,
   (o) substituted aryl,
   (p) heteroaryl, and
   (q) substituted heteroaryl. In one embodiment, the present invention provides compounds of formula (Q) above or a pharmaceutically acceptable salt, ester or prodrug thereof, wherein
V is carbonyl;
Y and Z are taken together define a group X, wherein X is ═N—C(O)R¹⁵, wherein R¹⁵ is selected from the group consisting of
(1) C₁-C₁₂-alkyl,
(2) substituted C₁-C₁₂-alkyl,
(3) C₁-C₆-alkoxy, and
(4) amino;
Ra is selected from the group consisting of hydrogen, C₂-C₁₂-alkyl, substituted C₁-C₁₂-alkyl, C₂-C₄-alkenyl, and C₂-C₄-alkynyl;
Rb is hydrogen or halogen;
Rc is hydrogen or —C(O)CH₃;
Rd is ethyl;
Re is hydroxyl; and
R is selected from the group consisting of
(1) ═O;
(2)=CH(Rk), wherein Rk is a cis or trans substituent selected from the group consisting of
   (a) hydrogen,
   (b) halogen, wherein said halogen is Br,
   (c) C₂-C₁₂-alkenyl substituted with aryl,
   (d) C₂-C₁₂-alkenyl substituted with substituted aryl,
   (e) C₂-C₁₂-alkenyl substituted with heteroaryl,
   (f) C₂-C₁₂-alkynyl substituted with aryl,
   (g) C₂-C₁₂-alkynyl substituted with heteroaryl,
   (h) C₂-C₁₂-alkynyl substituted with substituted heteroaryl,
   (i) aryl,
   (j) substituted aryl, and
   (k) heteroaryl; and
   (3) ═N—O-(Rm), wherein Rm is selected from the group consisting of
   (a) C₁-C₁₂-alkyl substituted with substituted C₁-C₁₂-alkenyl,
   (b) C₁-C₁₂-alkyl substituted with aryl,
   (c) C₁-C₁₂-alkyl substituted with substituted aryl,
   (d) C₁-C₁₂-alkyl substituted with heteroaryl,
   (e) C₁-C₁₂-alkyl substituted with substituted heteroaryl, and
   (f) aryl.

In another embodiment, the present invention provides compounds of formula (Q) above or a pharmaceutically acceptable salt, ester or prodrug thereof, wherein
V is carbonyl;
Y and Z are taken together define a group X, wherein X is ═N—C(O)R¹⁵, wherein R¹⁵ is selected from the group consisting of
(1) C₁-C₁₂-alkyl, wherein said C₁-C₁₂-alkyl is methyl or ethyl;
(2) substituted C₁-C₁₂-alkyl, wherein said substituted C₁-C₁₂-alkyl is —CH₂OH, —CH₂OC(O)CH₃, —CH₂OCH₃, or —CH₂NH₂;
(3) C₁-C₆-alkoxy, wherein said C₁-C₆-alkoxy is —OCH₃; and
(4) amino;
Ra is selected from the group consisting of hydrogen, C₂-C₁₂-alkyl, substituted C₁-C₁₂-alkyl, C₂-C₄-alkenyl, and C₂-C₄-alkynyl;
Rb is hydrogen or fluorine;
Rc is hydrogen or —C(O)CH₃;
Rd is ethyl;
Re is hydroxyl; and
R is selected from the group consisting of
(1) ═O;
(2)=CH(Rk), wherein Rk is a cis or trans substituent selected from the group consisting of
   (a) hydrogen,
   (b) halogen, wherein said halogen is Br,
   (c) C₂-C₁₂-alkenyl substituted with aryl, wherein said C₂-C₁₂-alkenyl substituted with aryl is —CH═CH(C₆H₅),
   (d) C₂-C₁₂-alkenyl substituted with substituted aryl, wherein said C₂-C₁₂-alkenyl substituted with substituted aryl is 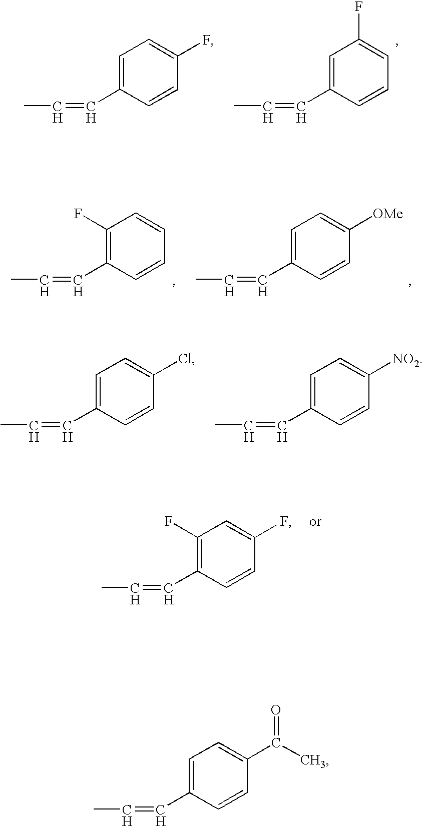
   (e) C₂-C₁₂-alkenyl substituted with heteroaryl, wherein said C₂-C₁₂-alkenyl substituted with heteroaryl is 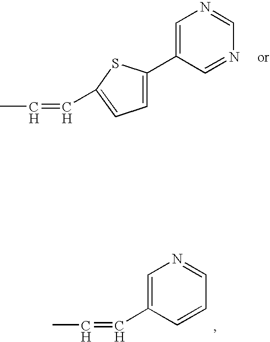
   (f) C₂-C₁₂-alkynyl substituted with aryl, wherein said C₂-C₁₂-alkynyl substituted with aryl is 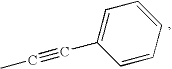
   (g) C₂-C₁₂-alkynyl substituted with heteroaryl, wherein said C₂C₁₂-alkynyl substituted with heteroaryl is 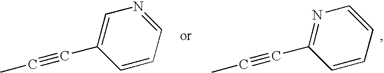
   (h) C₂-C₁₂-alkynyl substituted with substituted heteroaryl, wherein said C₂-C₁₂-alkynyl substituted with substituted heteroaryl is 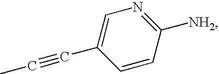
   (i) aryl, wherein said aryl is —C₆H₅, 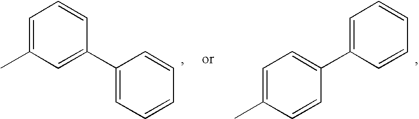
   (j) substituted aryl, wherein said substituted aryl is 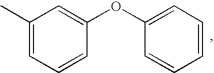
   (k) heteroaryl, wherein said heteroaryl is or 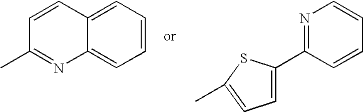
   and
(3) ═N—O-(Rm), wherein Rm is selected from the group consisting of
   (a) C₁-C₁₂-alkyl substituted with substituted C₁-C₁₂-alkenyl, wherein said C₁-C₁₂-alkyl substituted with substituted C₁-C₁₂-alkenyl is —CH₂—CH═CH(C₆H₅), (b) C₁-C₁₂-alkyl substituted with aryl, wherein said C₁-C₁₂-alkyl substituted with aryl is —CH₂(C₆H₅), —CH₂CH₂(C₆H₅), or —CH₂CH₂CH₂(C₆H₅),
   (c) C₁-C₁₂-alkyl substituted with substituted aryl, wherein said C₁-C₁₂-alkyl substituted with substituted aryl is 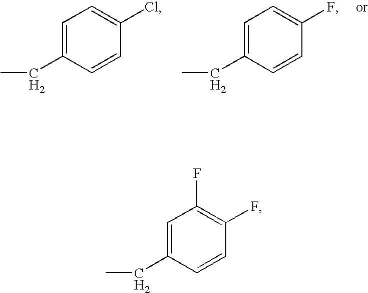
   (d) C₁-C₁₂-alkyl substituted with heteroaryl, wherein said C₁-C₁₂-alkyl substituted with heteroaryl is 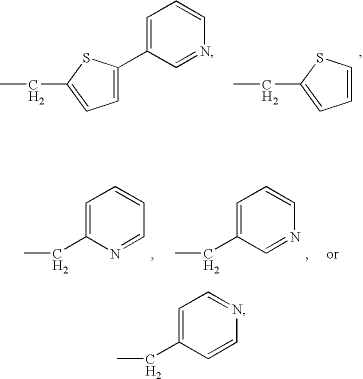
   (e) C₁-C₁₂-alkyl substituted with substituted heteroaryl, wherein said C₁-C₁₂-alkyl substituted with substituted heteroaryl is 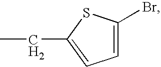
   and
   (f) aryl, wherein said aryl is —C₆H₅.

In some embodiments, the invention provides compounds wherein Ra is ethyl. In other embodiments, the invention provides compounds wherein Ra is vinyl. In still other embodiments, the invention provides compounds wherein Ra is acetylene.

In an embodiment, the invention provides compounds having the structure of the following formula 693:

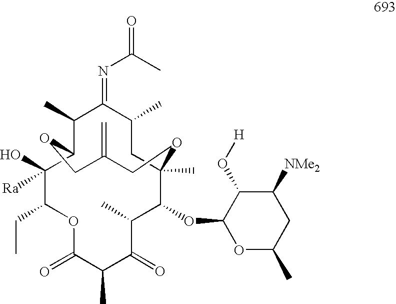

In another embodiment, the invention provides compounds having the structure of the following formula 308:

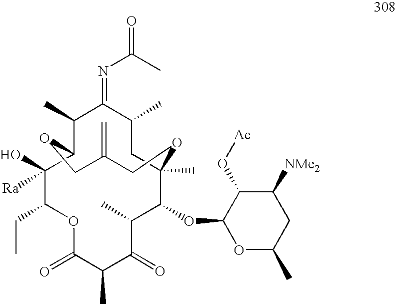

In another embodiment, the invention provides compounds having the structure of the following formula 218:

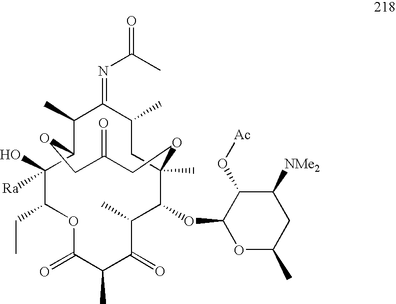

In another embodiment, the invention provides compounds having the structure of the following formula 589:

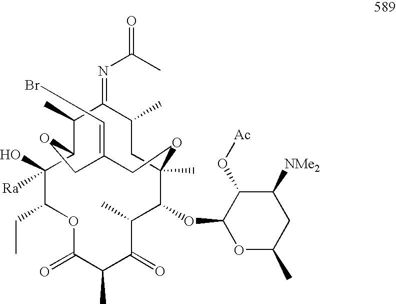

In another embodiment, the invention provides compounds having the structure of the following formula 879:

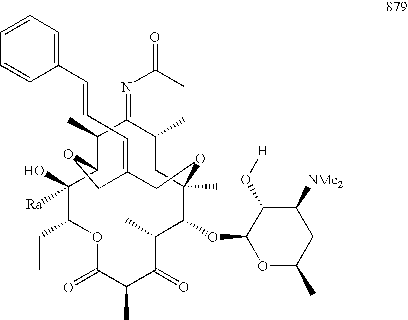

In another embodiment, the invention provides compounds having the structure of the following formula 400:

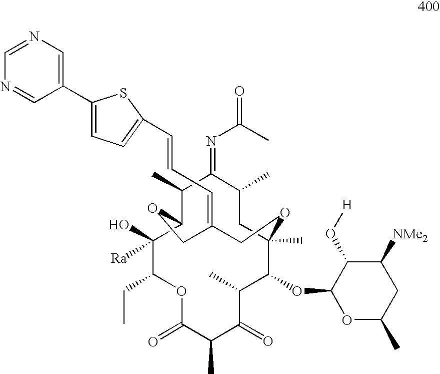

In another embodiment, the invention provides compounds having the structure of the following formula 539:

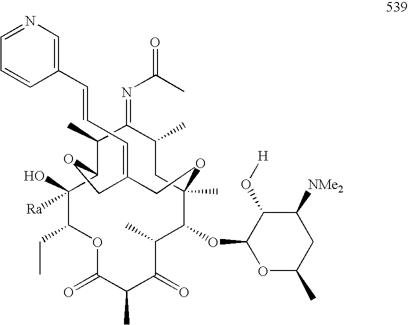

In another embodiment, the invention provides compounds having the structure of the following formula 643:

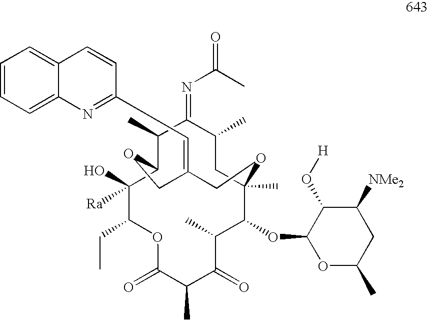

In another embodiment, the invention provides compounds having the structure of the following formula 580:

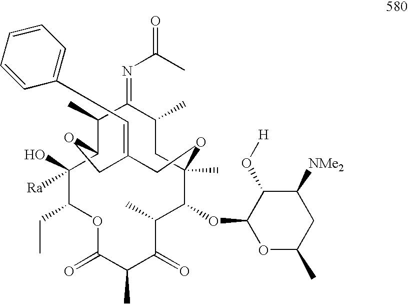

In another embodiment, the invention provides compounds having the structure of the following formula 958:

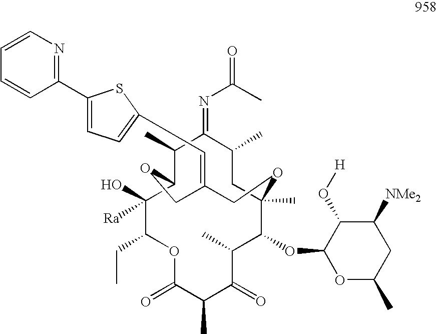

In another embodiment, the invention provides compounds having the structure of the following formula 015:

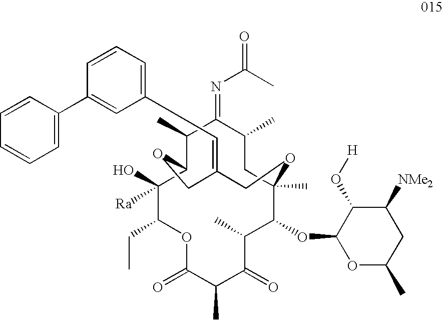

In another embodiment, the invention provides compounds having the structure of the following formula 174:

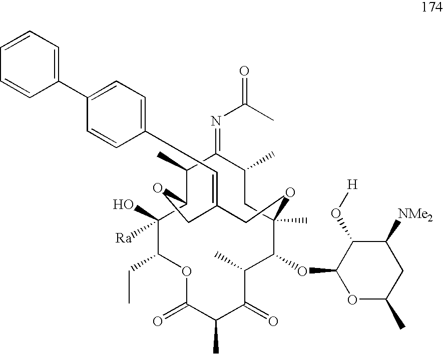

In another embodiment, the invention provides compounds having the structure of the following formula 264:

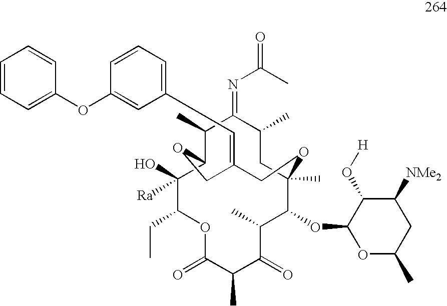

In another embodiment, the invention provides compounds having the structure of the following formula 413:

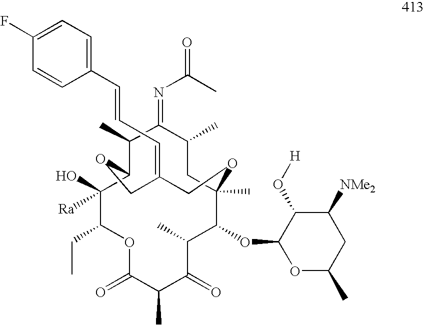

In another embodiment, the invention provides compounds having the structure of the following formula 598:

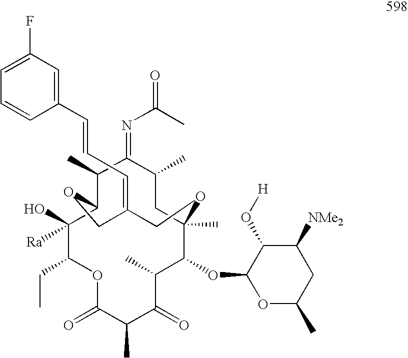

In another embodiment, the invention provides compounds having the structure of the following formula 873:

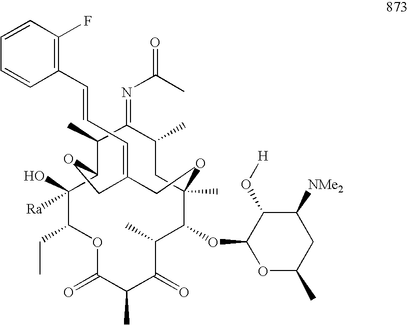

In another embodiment, the invention provides compounds having the structure of the following formula 253:

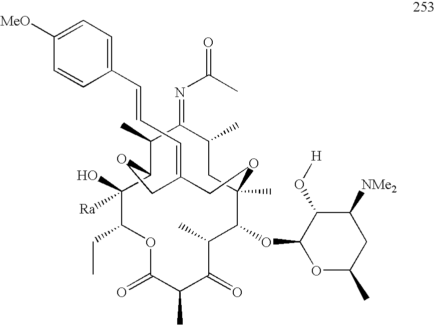

In another embodiment, the invention provides compounds having the structure of the following formula 271:

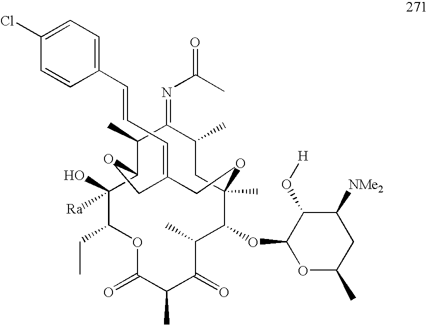

In another embodiment, the invention provides compounds having the structure of the following formula 202:

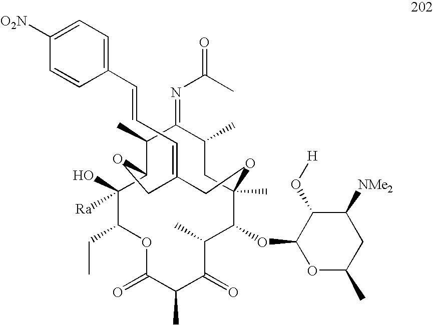

In another embodiment, the invention provides compounds having the structure of the following formula 952:

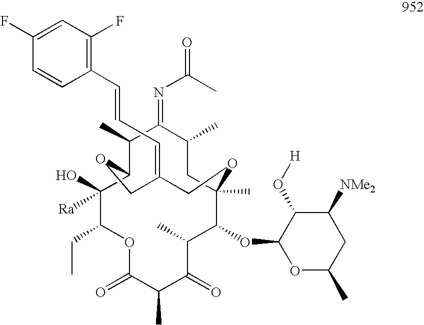

In another embodiment, the invention provides compounds having the structure of the following formula 608:

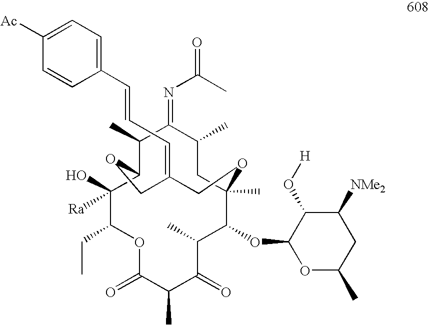

In another embodiment, the invention provides compounds having the structure of the following formula 668:

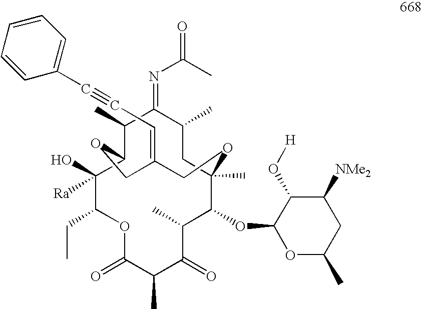

In another embodiment, the invention provides compounds having the structure of the following formula 926:

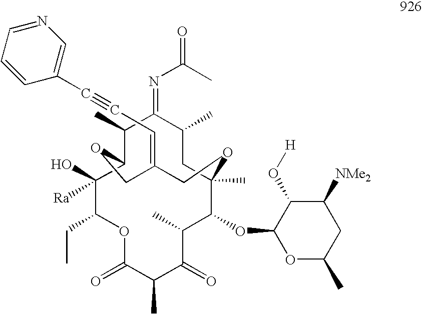

In another embodiment, the invention provides compounds having the e of the following formula 026:

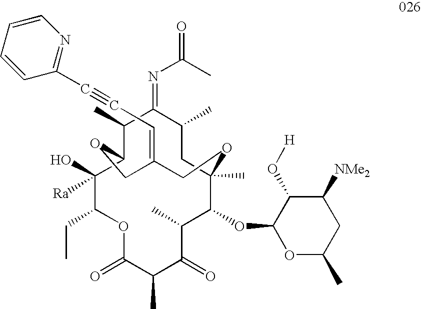

In another embodiment, the invention provides compounds having the of the following formula 687:

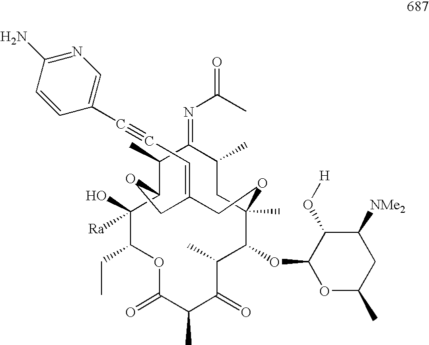

In another embodiment, the invention provides compounds having the structure of the following formula 592:

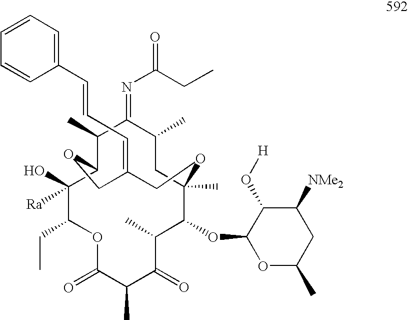

In another embodiment, the invention provides compounds having the structure of the following formula 941:

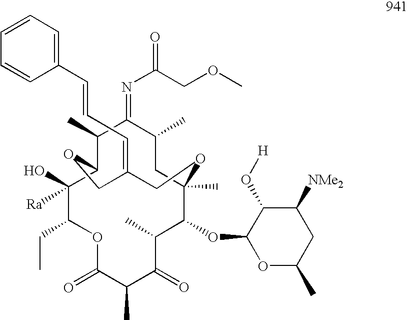

In another embodiment, the invention provides compounds having the structure of the following formula 676:

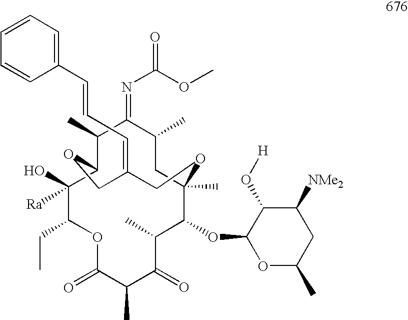

In another embodiment, the invention provides compounds having the structure of the following formula 367:

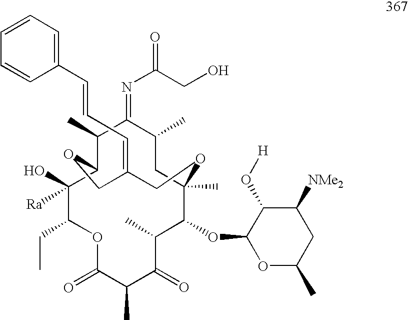

In another embodiment, the invention provides compounds having the structure of the following formula 771:

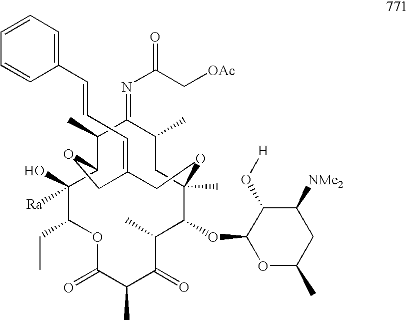

In another embodiment, the invention provides compounds having the structure of the following formula 052:

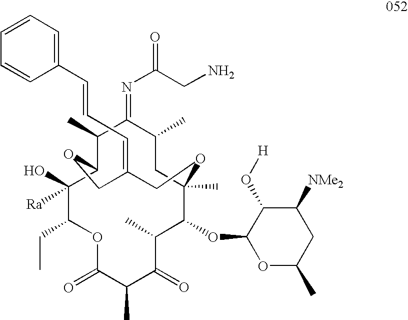

In another embodiment, the invention provides compounds having the structure of the following formula 668:

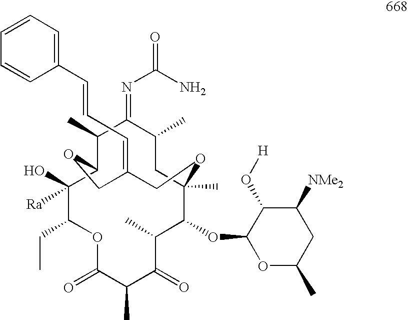

In another embodiment, the invention provides compounds having the structure of the following formula 932:

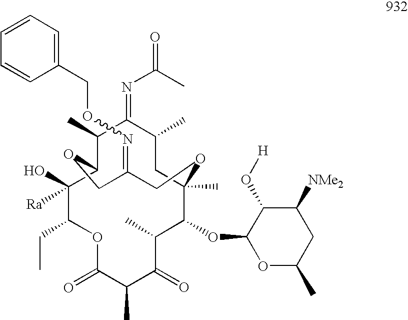

In another embodiment, the invention provides compounds having the structure of the following formula 230:

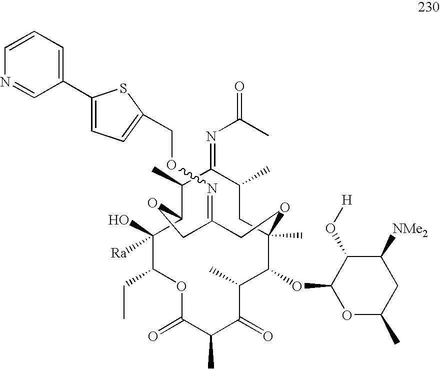

In another embodiment, the invention provides compounds having the structure of the following formula 911:

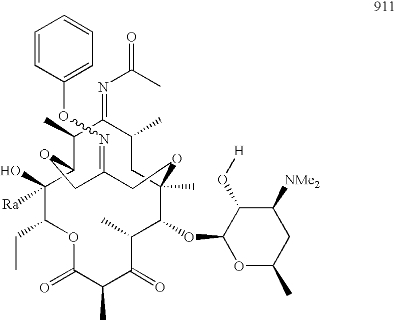

In another embodiment, the invention provides compounds having the structure of the following formula 841:

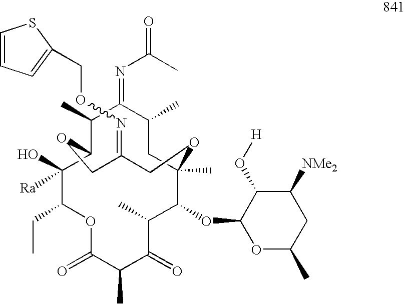

In another embodiment, the invention provides compounds having the structure of the following formula 629:

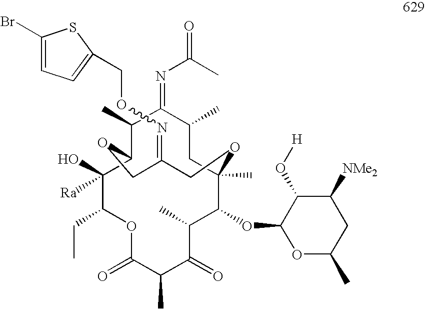

In another embodiment, the invention provides compounds having the structure of the following formula 512:

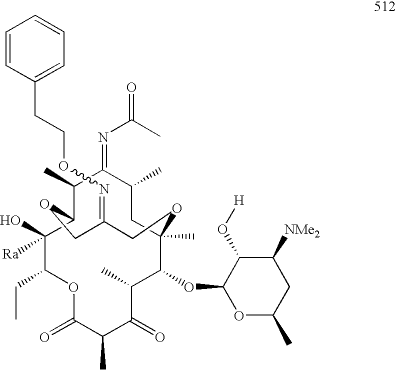

In another embodiment, the invention provides compounds having the structure of the following formula 914:

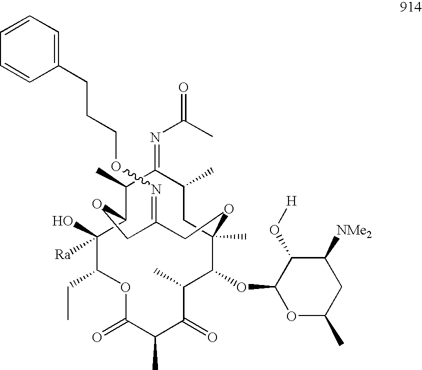

In another embodiment, the invention provides compounds having the e of the following formula 664:

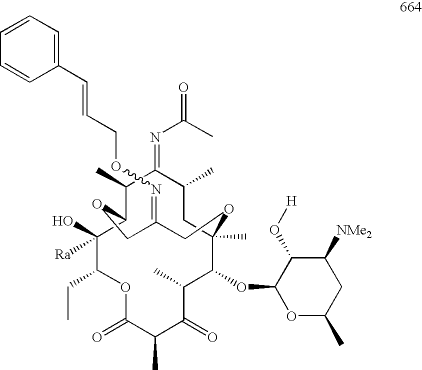

In another embodiment, the invention provides compounds having the e of the following formula 889:

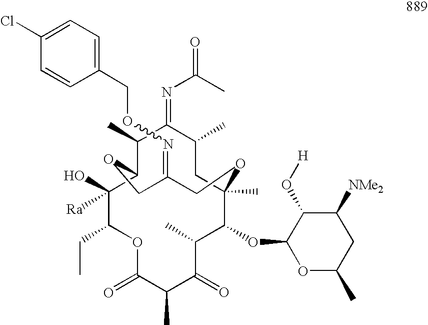

In another embodiment, the invention provides compounds having the structure of the following formula 568:

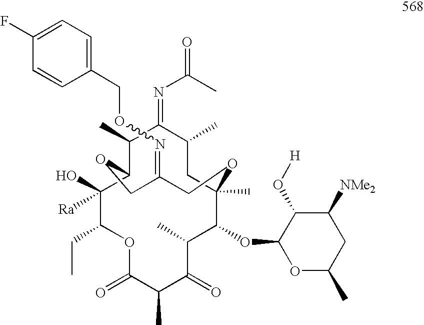

In another embodiment, the invention provides compounds having the structure of the following formula 753:

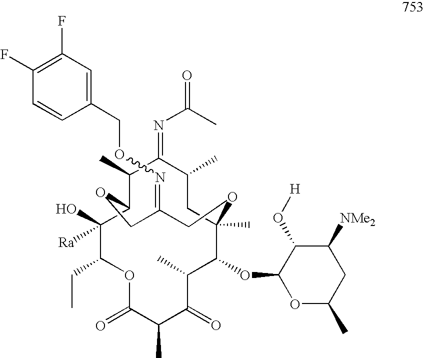

In another embodiment, the invention provides compounds having the structure of the following formula 459:

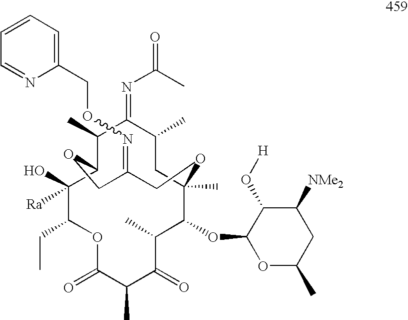

In another embodiment, the invention provides compounds having the structure of the following formula 220:

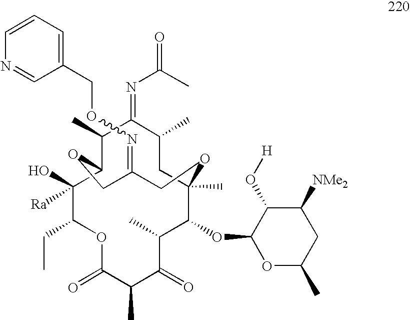

In another embodiment, the invention provides compounds having the structure of the following formula 311:

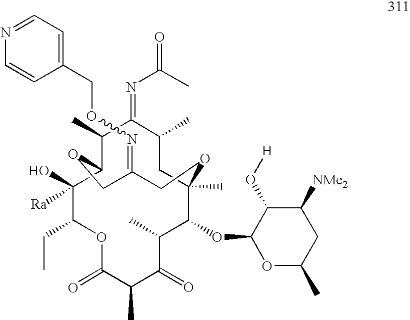

In yet another embodiment, the invention provides for a pharmaceutical composition comprising a compound presented herein, pharmaceutically acceptable salts, esters, or prodrugs thereof, and a pharmaceutically acceptable carrier.

In yet another embodiment, the invention provides for a method of treating bacterial infection in a mammalian patient in need thereof comprising administering to said patient a pharmaceutical composition comprising a therapeutically effective amount of a compound presented herein, or a pharmaceutically acceptable salt, ester, or prodrug thereof, and a pharmaceutically acceptable carrier. In a preferred embodiment, the mammalian patient is a human or a domesticated animal, such as dogs, cats, horses, cows, cattle, sheep, pigs, goats, lamas, donkeys, and mice.

The invention also provides for use of the compounds of the present invention or the pharmaceutically acceptable salts thereof, esters thereof, stereoisomers thereof, tautomers thereof, hydrates thereof, or solvates thereof in the manufacture of a medicament for the treatment or prophylaxis of a bacterial infection.

As used throughout this specification and the appended claims, the following terms have the meanings specified.

The term “alkyl” refers to saturated, straight- or branched-chain hydrocarbon groups that do not contain heteroatoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. Alkyl also includes cyclic alkyl groups such as cyclopropyl, cyclobutyl, cyclopentyl, cyclohexyl, cycloheptyl, and cyclooctyl and such rings substituted with straight and branched chain alkyl groups as defined above. Thus the phrase alkyl groups includes primary alkyl groups, secondary alkyl groups, and tertiary alkyl groups. Preferred alkyl groups include straight and branched chain alkyl groups and cyclic alkyl groups having 1 to 12 carbon atoms. Preferred straight chain alkyl groups include ethyl.

The phrase “substituted alkyl” refers to an alkyl group as defined above in which one or more bonds to a carbon(s) or hydrogen(s) are replaced by a bond to non-hydrogen and non-carbon atoms such as, but not limited to, a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups also include groups in which one or more bonds to a carbon(s) or hydrogen(s) atom is replaced by a higher-order bond (e.g., a double- or triple-bond) to a heteroatom, such as oxygen in oxo, carbonyl, carboxyl, and ester groups; or nitrogen in groups such as imines, oximes, hydrazones, and nitriles. Substituted alkyl groups further include alkyl groups in which one or more bonds to a carbon(s) or hydrogen(s) atoms is replaced by a bond to an aryl, heterocyclyl group, or cycloalkyl group. Preferred substituted alkyl groups include, among others, alkyl groups in which one or more bonds to a carbon or hydrogen atom is/are replaced by one or more bonds to fluorine atoms. Another preferred substituted alkyl group is the trifluoromethyl group and other alkyl groups that contain the trifluoromethyl group. Other preferred substituted alkyl groups include those in which one or more bonds to a carbon or hydrogen atom is replaced by a bond to an oxygen atom such that the substituted alkyl group contains a hydroxyl, alkoxy, or aryloxy group. Still other preferred substituted alkyl groups include alkyl groups that have an amine, or a substituted or unsubstituted alkylamine, dialkylamine, arylamine, (alkyl)(aryl)amine, diarylamine, heterocyclylamine, diheterocyclylamine, (alkyl)(heterocyclyl)amine, or (aryl)(heterocyclyl)amine group.

The terms “C₁-C₃-alkyl”, “C₁-C₆-alkyl”, and “C₁-C₁₂-alkyl” as used herein refer to saturated, straight- or branched-chain hydrocarbon radicals derived from a hydrocarbon moiety containing between one and three, one and six, and one and twelve carbon atoms, respectively, by removal of a single hydrogen atom. Examples of C₁-C₃-alkyl radicals include methyl, ethyl, propyl, and isopropyl. Examples of C₁-C₆-alkyl radicals include, but are not limited to, methyl, ethyl, propyl, isopropyl, n-butyl, tert-butyl, neopentyl and n-hexyl. Examples of C₁-C₁₂-alkyl radicals include, but are not limited to, all the foregoing examples as well as n-heptyl, n-octyl, n-nonyl, n-decyl, n-undecyl and n-dodecyl.

The term “C₁-C₆-alkoxy” as used herein refers to a C₁-C₆-alkyl group, as previously defined, attached to the parent molecular moiety through an oxygen atom. Examples of C₁-C₆-alkoxy include, but are not limited to, methoxy, ethoxy, propoxy, isopropoxy, n-butoxy, tert-butoxy, neopentoxy, and n-hexoxy.

The term “C₂-C₁₂-alkenyl” denotes a monovalent group derived from a hydrocarbon moiety containing from two to twelve carbon atoms and having at least one carbon-carbon double bond by the removal of two hydrogen atoms. Alkenyl groups include, for example, ethenyl, propenyl, butenyl, 1-methyl-2-buten-1-yl, and the like.

The phrase “substituted alkenyl” refers to an alkenyl group as defined above in which one or more bonds to a carbon(s) or hydrogen(s) are replaced by one or more bonds to non-hydrogen and non-carbon atoms such as, but not limited to, a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups. Substituted alkenyl groups also include groups in which one or more bonds to a carbon(s) or hydrogen(s) atom is replaced by a higher-order bond (e.g., a double- or triple-bond) to a heteroatom such as oxygen in oxo, carbonyl, carboxyl, and ester groups; or nitrogen in groups such as imines, oximes, hydrazones, and nitriles. Substituted alkenyl groups further include groups in which one or more bonds to a carbon(s) or hydrogen(s) atoms is replaced by a bond to an aryl, heterocyclyl group, or cycloalkyl group. For example, in the case of ethenyl, combinations in which the CH₂═CH— group is monosubstituted can be referred to as vinyl groups, such as in vinyl chloride (CH₂═CH—Cl) and methyl vinyl ether (CH₂═CH—OCH₃).

The term “C₂-C₁₂-alkynyl” as used herein refers to a monovalent group derived from a straight or branched chain hydrocarbon moiety containing from two to twelve carbon atoms and having at least one carbon-carbon triple bond, typically formed by the removal of four hydrogen atoms. Representative alkynyl groups include ethynyl, i.e. acetylene, propynyl, 2-propynyl, 1-butynyl, 2-butynyl, 3-butynyl, 2-(3-butyn)-yl, and the like.

The term 14-member macrolide antibiotics used herein include the natural products erythromycin, narbomycin, lakamycin, and oleandomycin, as well as derivatives such as roxithromycin, clarithromycin, dirithromycin, flurithromycin, and the ketolides (telithromycin, HMR 3004, TE-802, TE-810, ABT 773).

The term “alkylene” denotes a divalent group derived from a straight or branched chain saturated hydrocarbon by the removal of two hydrogen atoms, for example methylene, 1,2-ethylene, 1,1-ethylene, 1,3-propylene, 2,2-dimethyl-propylene, and the like.

The term “C₁-C₃-alkylamino” as used herein refers to one or two C₁-C₃-alkyl groups, as previously defined, which may be the same or different, attached to the parent molecular moiety through a nitrogen atom. Examples of C₁-C₃-alkylamino include, but are not limited to methylamino, dimethylamino, ethylamino, diethylamino, n-propylamino, di-n-propylamino, 2-propylamino, methylethylamino, and the like.

The term “oxo” denotes a group wherein two hydrogen atoms on a single carbon atom in an alkyl group as defined above are replaced with a single oxygen atom (i.e. a carbonyl group).

The term “aryl” as used herein refers to a mono-, bi-, or tri-cyclic carbocyclic ring system having one, two, or three aromatic rings including, but not limited to, phenyl, naphthyl, tetrahydronaphthyl, indanyl, indenyl and the like. In addition, aryl groups may comprise fused aromatic rings. Representative aryl groups comprise from 3 to 12 carbon atoms. Preferable aryl groups include, but are not limited to, aryl groups of C₃-C₁₂, such as C₄-C₁₀ and C₆-C₈. Aryl groups (including bi- or tricyclic aryl groups) can be unsubstituted or substituted with one, two or three substituents independently selected from loweralkyl, substituted loweralkyl, haloalkyl, alkoxy, thioalkoxy, amino, alkylamino, dialkylamino, acylamino, cyano, hydroxy, halo, mercapto, nitro, carboxaldehyde, carboxy, alkoxycarbonyl and carboxamide. In addition, substituted aryl groups include tetrafluorophenyl and pentafluorophenyl.

The term “C₃-C₁₂-cycloalkyl” denotes a monovalent group derived from a monocyclic or bicyclic saturated carbocyclic ring compound by the removal of a single hydrogen atom. Examples include cyclopropyl, cyclobutyl, cyclopentyl, cyclohexyl, bicyclo[2.2.1 I]heptyl, and bicyclo[2.2.2]octyl.

The terms “halo” and “halogen” as used herein refer to an atom selected from fluorine, chlorine, bromine and iodine.

The term “alkylamino” refers to a group having the structure —NHR′ wherein R′ is alkyl, as previously defined. Examples of alkylamino include, but are not limited to, methylamino, ethylamino, iso-propylamino.

The term “dialkylamino” refers to a group having the structure —NR′R″ wherein R′ and R″ are independently selected from alkyl, as previously defined. Additionally, R′ and R″ taken together may optionally be —(CH₂)ₖ— where k is an integer of from 2 to 6. Examples of dialkylamino include, but are not limited to, dimethylamino, diethylamino, diethylaminocarbonyl, methylethylamino, methyl-propylamino, and piperidino.

The term “oxime” refers to a group having the structure ═N—O—R wherein R is a cis or trans substituent. Preferably, R is alkyl, substituted alkyl, aryl, substituted aryl, heteroaryl, or substituted heteroaryl.

The term “haloalkyl” denotes an alkyl group, as defined above, having one, two, or three halogen atoms attached thereto and is exemplified by such groups as chloromethyl, bromoethyl, trifluoromethyl and the like.

The term “alkoxycarbonyl” represents an ester group; i.e. an alkoxy group, attached to the parent molecular moiety through a carbonyl group such as methoxycarbonyl, ethoxycarbonyl, and the like.

The term “thioalkyl” refers to an alkyl group as previously defined attached to the parent molecular moiety through a sulfur atom.

The term “carboxaldehyde” as used herein refers to a group of formula —CHO.

The term “carboxy” as used herein refers to a group of formula —CO₂H.

The term “carboxamide” as used herein refers to a group of formula —CONHR′R″ wherein R′ and R″ are independently selected from hydrogen or alkyl, or R′ and R″ taken together may optionally be —(CH₂)ₖ—, wherein k is an integer of from 2 to 6.

The term “heteroaryl”, as used herein, refers to a cyclic or bicyclic aromatic radical having from five to ten ring atoms in each ring of which one atom of the cyclic or bicyclic ring is selected from S, O and N; wherein zero, one, or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and naphthyridinyl. Representative heteroaryl groups comprise from 3 to 12 carbon atoms. Preferable heteroaryl groups include, but are not limited to, aryl groups of C₃-C₁₂, such as C₄-C₁₀ and C₆-C₈. Representative examples of heteroaryl moieties include, but not limited to, pyridin-3-yl-1H-imidazol-1-yl, phenyl-1H-imidazol-1-yl, 3H-imidazo[4,5-b]pyridin-3-yl, quinolin-4-yl, 4-pyridin-3-yl-1H-imidazol-1-yl, quinolin-4-yl, quinolin-2-yl, 2-methyl-4-pyridin-3-yl-1H-imidazol-1-yl, 5-methyl-4-pyridin-3-yl-1H-imidazol-1-yl, 1H-imidazo[4,5-b]pyridin-1-yl, pyridin-3-ylmethyl, 3H-imidazo[4,5-b]pyridin-3-yl, 4-pyrimidin-5-yl-1H-imidazol-1-yl, 4-pyrazin-2-yl-1H-imidazol-1-yl, 4-pyridin-3-yl-1H-imidazol-1-yl, 4-pyridin-4-yl-1H-imidazol-1-yl, 4-(6-methylpyridin-3-yl)-1H-imidazol-1-yl, 4-(6-fluoropyridin-3-yl)-1H-imidazol-1-yl, 5-(3-aminophenyl)-1,3-thiazol-2-yl, 3-pyridin-3-ylphenoxy, 4-pyridin-3-ylphenoxy, 3H-imidazo[4,5-b]pyridin-3-yl, 4-phenyl-1H-imidazol-1-yl, 1H-pyrrolo[3,2-b]pyridin-1-yl, quinolin-3-yl, 2-methylquinolin-4-yl, trifluoromethyl)quinolin-4-yl, 8-(trifluoromethyl)quinolin-4-yl, 2-phenoxyethoxy, 4-pyridin-3-ylphenoxy, 3-pyridin-3-ylphenoxy, 5-phenyl-1,3-thiazole, 5-(2,4-difluorophenyl)-1,3-thiazol-2-yl, 5-(3-aminophenyl)-1,3-thiazol-2-yl, (3,3′-bipyridin-5-ylmethyl)(methyl)amino, (6-methylpyridin-3-yl)-1H-imidazol-1-yl, methyl(quinolin-3-ylmethyl)amino, 3-phenylisoxazol-5-yl, 3-(4-methylphenyl)isoxazol-5-yl and the like.

The term “heterocycloalkyl” as used herein, refers to a non-aromatic partially unsaturated or fully saturated 3- to 10-membered ring system, which includes single rings of 3 to 8 atoms in size and bi- or tri-cyclic ring systems which may include aromatic six-membered aryl or heteroaryl rings fused to a non-aromatic ring. These heterocycloalkyl rings include those having from one to three heteroatoms independently selected from oxygen, sulfur and nitrogen, in which the nitrogen and sulfur heteroatoms may optionally be oxidized and the nitrogen heteroatom may optionally be quaternized.

Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “heteroarylalkyl” as used herein, refers to a heteroaryl group as defined above attached to the parent molecular moiety through an alkylene group wherein the alkylene group is of one to four carbon atoms.

“Hydroxy-protecting group”, as used herein, refers to an easily removable group which is known in the art to protect a hydroxyl group against undesirable reaction during synthetic procedures and to be selectively removable. The use of hydroxy-protecting groups is well known in the art for protecting groups against undesirable reactions during a synthetic procedure and many such protecting groups are known, cf, for example, T. H. Greene and P. G. M. Wuts, Protective Groups in Organic Synthesis, 2nd edition, John Wiley & Sons, New York (1991). Examples of hydroxy-protecting groups include, but are not limited to, methylthiomethyl, tert-dimethylsilyl, tert-butyldiphenylsilyl, ethers such as methoxymethyl, and esters including acetyl, benzoyl and the like.

The term “ketone protecting group”, as used herein, refers to an easily removable group which is known in the art to protect a ketone group against undesirable reaction during synthetic procedures and to be selectively removable. The use of ketone-protecting groups is well known in the art for protecting groups against undesirable reaction during a synthetic procedure and many such protecting groups are known, cf., for example, T. H. Greene and P. G. M. Wuts, Protective Groups in Organic Synthesis, 2nd edition, John Wiley & Sons, New York (1991). Examples of ketone-protecting groups include, but are not limited to, ketals, oximes, O-substituted oximes for example O-benzyl oxime, O-phenylthiomethyl oxime, 1-isopropoxycyclohexyl oxime, and the like.

The term “protected-hydroxyl” refers to a hydroxyl group protected with a hydroxyl protecting group, as defined above, including benzoyl (Bz), acetyl (Ac), trimethylsilyl (TMS), triethylsilyl (TES), and methoxymethyl groups, for example.

The term “substituted aryl” as used herein refers to an aryl group as defined herein substituted by independent replacement of one, two or three of the hydrogen atoms thereon with Cl, Br, F, I, OH, CN, C₁-C₃-alkyl, C₁-C₆-alkoxy, C₁-C₆alkoxy substituted with aryl, haloalkyl, thioalkyl, thioalkoxy, amino, alkylamino, dialkylamino, mercapto, nitro, carboxaldehyde, carboxy, alkoxycarbonyl and carboxamide. In addition, any one substituent may be an aryl, heteroaryl, or heterocycloalkyl group.

The term “substituted heteroaryl” as used herein refers to a heteroaryl group as defined herein substituted by independent replacement of one, two or three of the hydrogen atoms thereon with Cl, Br, F, I, OH, CN, C₁-C₃-alkyl, C₁-C₆-alkoxy, C₁-C₆-alkoxy substituted with aryl, haloalkyl, thioalkoxy, amino, alkylamino, dialkylamino, mercapto, nitro, carboxaldehyde, carboxy, alkoxycarbonyl and carboxamide. In addition, any one substituent may be an aryl, heteroaryl, or heterocycloalkyl group.

The term “substituted heterocycloalkyl” as used herein, refers to a heterocycloalkyl group, as defined above, substituted by independent replacement of one, two or three of the hydrogen atoms thereon with Cl, Br, F, I, OH, CN, C₁-C₃-alkyl, C₁-C₆-alkoxy, C₁-C₆-alkoxy substituted with aryl, haloalkyl, thioalkyl, thioalkoxy, amino, alkylamino, dialkylamino, mercapto, nitro, carboxaldehyde, carboxy, alkoxycarbonyl and carboxamide. In addition, any one substituent may be an aryl, heteroaryl, or heterocycloalkyl group.

Numerous asymmetric centers may exist in the compounds of the present invention. Except where otherwise noted, the present invention contemplates the various stereoisomers and mixtures thereof. Accordingly, whenever a bond is represented by a wavy line, it is intended that a mixture of stereo-orientations or an individual isomer of assigned or unassigned orientation may be present.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Representative examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

Pharmaceutical compositions of the present invention comprise a therapeutically effective amount of a compound of the present invention formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and other animals orally, rectally, parenterally, intracisternally, intravaginally, intraperitoneally, topically (as by powders, ointments, or drops), bucally, or as an oral or nasal spray, or a liquid aerosol or dry powder formulation for inhalation.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions of the invention may also be formulated for delivery as a liquid aerosol or inhalable dry powder. Liquid aerosol formulations may be nebulized predominantly into particle sizes that can be delivered to the terminal and respiratory bronchioles where bacteria reside in patients with bronchial infections, such as chronic bronchitis and pneumonia. Pathogenic bacteria are commonly present throughout airways down to bronchi, bronchioli and lung parenchema, particularly in terminal and respiratory bronchioles. During exacerbation of infection, bacteria can also be present in alveoli. Liquid aerosol and inhalable dry powder formulations are preferably delivered throughout the endobronchial tree to the terminal bronchioles and eventually to the parenchymal tissue.

Aerosolized formulations of the invention may be delivered using an aerosol forming device, such as a jet, vibrating porous plate or ultrasonic nebulizer, preferably selected to allow the formation of a aerosol particles having with a mass medium average diameter predominantly between 1 to 5 microns. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulize the formulation of the invention into aerosol particle size predominantly in the size range from 1-5 microns. Predominantly in this application means that at least 70% but preferably more than 90% of all generated aerosol particles are within 1-5 micron range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AeroNeb and AeroDose vibrating porous plate nebulizers (AeroGen, Inc., Sunnyvale, Calif.), Sidestream nebulizers (Medic-Aid Ltd., West Sussex, England), Pari LC and Pari LC Star jet nebulizers (Pari Respiratory Equipment, Inc., Richmond, Va.), and Aerosonic (DeVilbiss Medizinische Produkte (Deutschland) GmbH, Heiden, Germany) and UltraAire (Omron Healthcare, Inc., Vernon Hills, Ill.) ultrasonic nebulizers.

Compounds of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present invention, bacterial infections are treated or prevented in a patient such as a human or lower mammal by administering to the patient a therapeutically effective amount of a compound of the invention, in such amounts and for such time as is necessary to achieve the desired result. By a “therapeutically effective amount” of a compound of the invention is meant a sufficient amount of the compound to treat bacterial infections, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. The total daily dose of the compounds of this invention administered to a human or other mammal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 2000 mg of the compound(s) of this invention per day in single or multiple doses.

The foregoing may be better understood by reference to the following examples which are presented for illustration and not to limit the scope of the inventive concepts.

### DESCRIPTION OF THE PREFERRED EMBODIMENTS

The following examples are provided for illustrative purposes and are not to be construed to limit the scope of the claims in any manner whatsoever.

### EXAMPLE 1

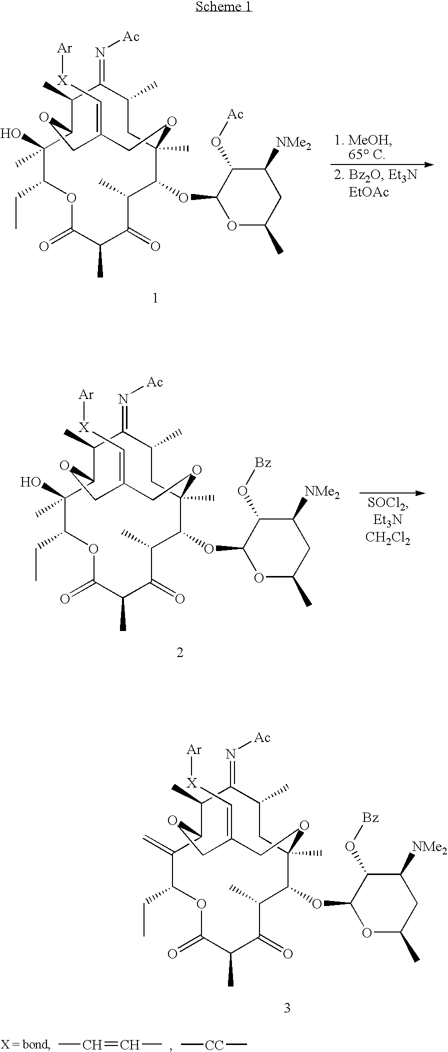 Preparation of Compound 2

Compound 1 is dissolved in methanol (0.1M) and heated to approximately 60-65° C. for about 2-24 hrs until all of the starting material is converted. The reaction is cooled to room temperature and concentrated in vacuo to yield a 4′-OH crude intermediate. The crude intermediate is purified by column chromatography (silica gel, MeOH/CH₂Cl₂/NH₄OH or hexanes/acetone/TEA).

To the 4′-OH crude intermediate in ethyl acetate or CH₂Cl₂ (0.1M), TEA (2 equiv.) and benzoic anhydride (2 equiv.) is added. The mixture is stirred at room temperature for approximately 2-24 hrs until the starting material is completely converted. The mixture is diluted with reaction solvent and washed twice with saturated aqueous NaHCO₃, brine, and dried with Na₂SO₄, and filtered. The filtrate is concentrated in vacuo and purified by column chromatography (silica gel, MeOH/CH₂Cl₂/NH₄OH or hexanes/acetone/TEA) to yield the 4′-OBz Compound 2.

Preparation of Compound 3

Compound 2 is dissolved in CH₂Cl₂ (0.1M) and cooled to 0° C. To this, TEA (4 equiv.) and SOCl₂ (1.2 equiv.) is added sequentially. The reaction is maintained at 0° C. for approximately 1-8 hrs. The reaction is quenched with saturated NaHCO₃, and the organic layer is washed three times with 5% KH₂PO₄, water and brine, dried over Na₂SO₄, and concentrated. The material is purified by column chromatography (silica gel, MeOH/CH₂Cl₂/NH₄OH or hexanes/acetone/TEA) to yield the 11,12-olefin Compound 3.

### EXAMPLE 2

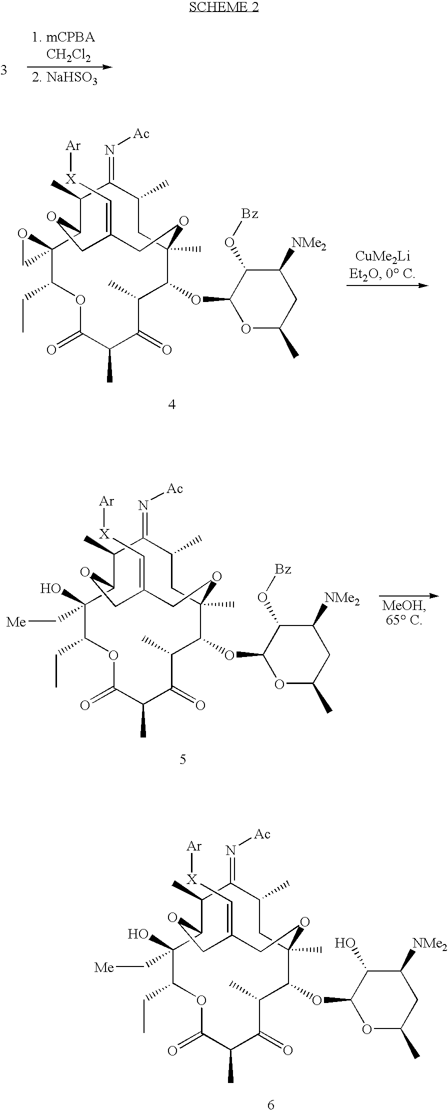 Preparation of Compound 4

To a 0° C. dichloromethane solution of Compound 3 (0.1M), 3-chloroperoxybenzoic acid (2.2 equiv) is added. The reaction is stirred at 0° C. for 30 minutes, then warmed to room temperature for about 2-8 hours. Cyclohexene (1.8 equiv) is added and the solution is stirred for an additional 15 minutes. The solution is diluted with dichloromethane and washed with saturated NaHCO₃ and brine, dried over Na₂SO₄, filtered, and concentrated. Purification by column chromatography (silica gel, MeOH/CH₂Cl₂/NH₄OH or hexanes/acetone/TEA) yields an epoxide, N-oxide crude intermediate.

To a tetrahydrofuran solution of the above epoxide, N-oxide crude intermediate (0.1M), an aqueous solution (0.5M) of sodium bisulfite (8 equiv) is added. The mixture is stirred at room temperature for approximately 8-18 hours. The solution is diluted with ethyl acetate and washed with saturated NaHCO₃ and brine, dried over Na₂SO₄, filtered, and concentrated. Purification by column chromatography (silica gel, MeOH/CH₂Cl₂/NH₄OH or hexanes/acetone/TEA) yields the epoxide, Compound 4.

Preparation of Compound 5

A flame dried 2-neck flask is cooled under argon. An internal thermocouple is inserted and CuBr dimethyl sulfide complex (5 equiv) is added. The system is evacuated under high vacuum and purged with argon three times. Diethyl ether (anhydrous, 0.05M in CuBr) is added and the heterogeneous solution is cooled in a −78° C. bath. Methyl lithium (10 equiv) is added via syringe while maintaining an internal temperature of ≦−60° C. The solution is held in a −78° C. bath for 10 minutes and then the bath is removed. Upon warming to approximately −20° C. to about 0° C., a homogeneous solution is obtained and the solution is then held at −30° C. Separately, a flame dried 2-neck flask is cooled under argon. Compound 4 is added and the system is evacuated under high vacuum and purged with argon three times. Diethyl ether is added (0.1 M). This solution is added via syringe to the above prepared cuprate solution (at −30° C.). The internal temperature during the addition is kept at ≦−10° C. The resultant solution is held at 0° C. for approximately 6-16 hours with stirring. The reaction is quenched with saturated aqueous NH₄Cl solution, while maintaining an internal temperature ≦−10° C. The solution is diluted with ethyl acetate and washed with saturated NH₄Cl and brine, dried over Na₂SO₄, filtered, and concentrated. Purification by column chromatography (silica gel, MeOH/CH₂Cl₂/NH₄OH or hexanes/acetone/TEA) yields the C₁₂-ethyl ketolide, Compound 5.

Preparation of Compound 6

Compound 5 is dissolved in methanol (0.1 M) and heated to approximately 60-65° C. for about 2-24 hrs until the starting material is completely converted. The reaction is cooled to room temperature and concentrated in vacuo. The crude material is purified by column chromatography (silica gel, MeOH/CH₂Cl₂/NH₄OH or hexanes/acetone/TEA) to yield Compound 6.

### EXAMPLE 3

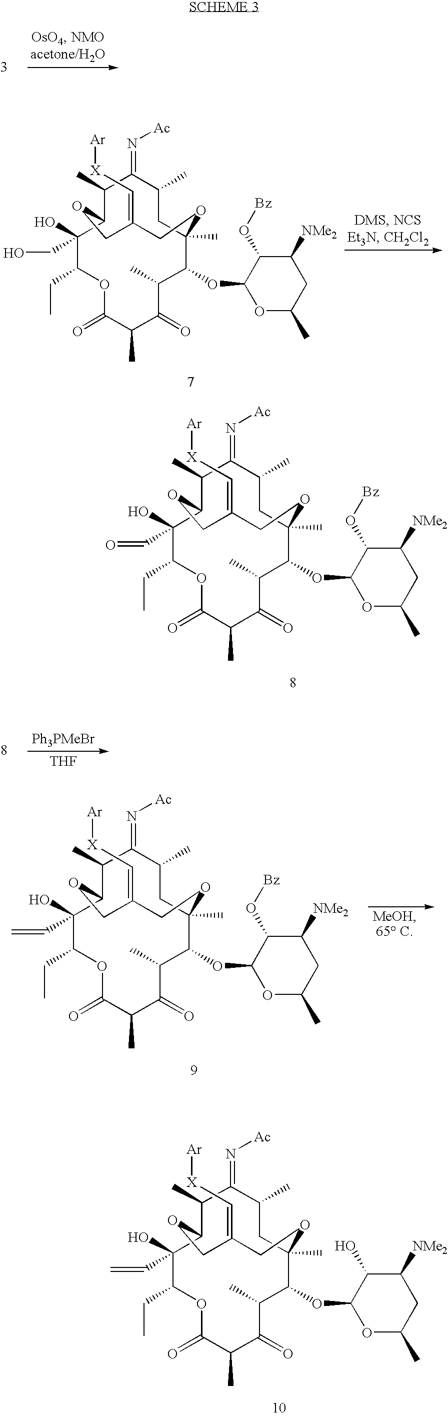 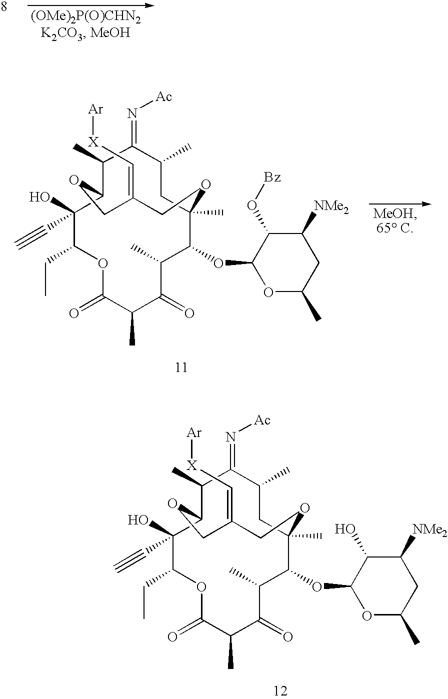 Preparation of Compound 7

To an acetone/H₂O (9:1) solution (0.1M) of Compound 3 at 0° C., N-methyl morpholine N-oxide (NMO) mono-dydrate (1.2 equiv) is added, followed by addition of 3% osmium tetroxide in tert-butanol. The solution is stirred at 0° C. to room temperature until the starting material is completely converted. The solution is diluted with ethyl acetate and cooled to 0° C. Upon cooling, saturated aqueous Na₂SO₃ is added and the solution is stirred for 10 minutes. The reaction is warmed to room temperature, diluted with ethyl acetate, washed with saturated NaHCO₃ and brine, dried over Na₂SO₄, filtered, and concentrated. Purification by column chromatography (silica gel, MeOH/CH₂Cl₂/NH₄OH or hexanes/acetone/TEA) yields ketolide Compound 7.

Preparation of Compound 8

To a solution of N-chlorosuccinimide, NCS, (1 equiv) in dichloromethane at 0° C., dimethyl sulfide (DMS, 1.2 equiv) is added. After stirring for five minutes, the solution is cooled to −20° C. Next, Compound 7 (0.4 equiv) in dichloromethane is added. The resulting solution is stirred at −20° C. for about 1.5-5 hours, at which time triethylamine (1 equiv) is added dropwise. After stirring at −20° C. for ten minutes, the solution is warmed to room temperature. The solution is then diluted with ethyl acetate and washed with saturated NaHCO₃ and brine, dried over Na₂SO₄, filtered, and concentrated. Purification by column chromatography (silica gel, MeOH/CH₂Cl₂/NH₄OH or hexanes/acetone/TEA) yields aldehyde Compound 8.

Preparation of Compound 9

To a solution of methyl triphenylphosphonium bromide (1 equiv) in tetrahydrofuran at −78° C., potassium bis(trimethylsilyl)amide (0.5M in toluene, 1 equiv) is added. The mixture is warmed slowly and stirred for 1 hour. After cooling the anion solution is cooled back to −78° C., a tetrahydrofuran solution (0.2M) of aldehyde Compound 8 (0.5 equiv) is added to the cooled solution. The cooling bath is removed and the reaction mixture is stirred for 4 hours or until the reaction is completed. The reaction is quenched with saturated aqueous NH₄Cl at 0° C. The solution is diluted with ethyl acetate, washed with saturated NH₄Cl and brine, dried over Na₂SO₄, filtered, and concentrated. Purification by column chromatography (silica gel, MeOH/CH₂Cl₂/NH₄OH or hexanes/acetone/TEA) yields the C₁₂-vinyl ketolide Compound 9.

Preparation of Compound 10

Compound 9 is dissolved in methanol (0.1 M) and heated to approximately 60-65° C. for about 2-24 hrs until the starting material is completely converted. The reaction is cooled to room temperature and concentrated in vacuo. The crude material is purified by column chromatography (silica gel, MeOH/CH₂Cl₂/NH₄OH or hexanes/acetone/TEA) to yield Compound 10.

Preparation of Compound 11

Compound 8 (1 equiv) is added to a 0° C. solution of Seyferth-Gilbert reagent, dimethyl diazomethylphosphonate, (1.20 equiv, synthesized according to references: J. Org. Chem. 1996, Vol. 61, p. 2540 and 1971, Vol. 36, p. 1379, the disclosures of which are incorporated herein by reference) in dry MeOH. Potassium carbonate (2.00 equiv) is added. The solution is stirred at 0° C. for 1.5 hours and then brought up to ambient temperature for approximately 1-4 hours. The reaction mixture is poured into ethyl acetate and washed sequentially with water and brine. The organic layer is dried over MgSO₄, filtered, and concentrated. Column chromatography (silica gel, MeOH/CH₂Cl₂/NH₄OH or hexanes/acetone/TEA) yields the desired alkyne Compound 11.

Preparation of Compound 12

Compound 11 is dissolved in methanol (0.1M) and heated to approximately 60-65° C. for about 2-24 hrs until the starting material is completely converted. The reaction is cooled to room temperature and concentrated in vacuo. The crude material is purified by column chromatography (silica gel, MeOH/CH₂Cl₂/NH₄OH or hexanes/acetone/TEA) to yield Compound 12.

Alternatively, the bridged analogs may also be prepared as shown below.

### EXAMPLE 4

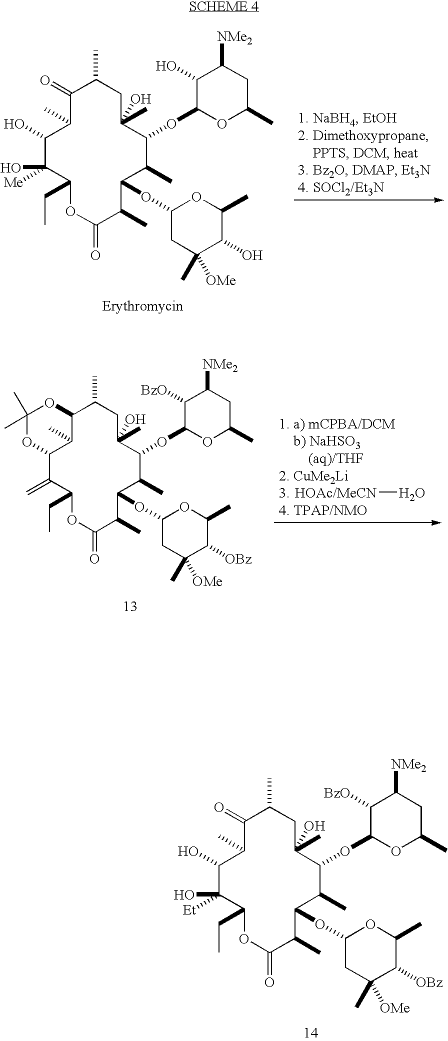 Preparation of Compound 13

To a solution of NaBH₄ (23 g) in 200 mL absolute ethanol at 0° C. under argon, an erythromycin solution (75 g in 200 mL absolute ethanol) was added over a 40 min period. The erythromycin was pre-dried azeotropically with benzene. The ice bath was removed and the reaction was stirred at room temperature for 18 hrs. CO₂ (dry ice) was bubbled through the reaction for 40 min, resulting in a very thick mixture. Triethanolamine ((HOCH₂CH₂)₃N, 25 mL) was added and the reaction was stirred overnight. Removal of solvent resulted in a solid/syrup. To this, 500 mL EtOAc and 500 mL of 5% aqueous KH₂PO₄ was added. The organic layer was separated, and the aqueous layer was thoroughly extracted with EtOAc and chloroform. The combined organic extracts were pooled, dried over Na₂SO₄, and filtered. The filtrate was concentrated in vacuo to give a white foam. This was triturated with dichloromethane/isopropylether to give three crops with total yield of 61 g (80%). Without further purification, this crude product was carried over to the next step. LCMS(ES) (MH⁺)=736.5.

The above purified material (27 g, white powder) was dissolved in 200 mL dichloromethane and 100 mL 2,2-dimethoxypropane (DMP). Pyridinium p-toluenesulfonic acid (PPTS, 27 g, 3 equiv.) was added and heated to approximately 50-55° C. Both the crude material and PPTS were pre-dried azeotropically using benzene. The reaction was monitored with TLC until almost all of the starting material was converted, approximately 18 hrs. The reaction was cooled and Et₃N (30 mL) was added. The reaction is then concentrated and re-dissolved in 400 mL CHCl₃ before being washed with 5% aqueous KH₂PO₄. The reaction was diluted with aqueous NH₄OH (1N) and brine, dried with Na₂SO₄ and concentrated in vacuo. Purification was performed on a silica gel flash column, and the product was eluted with 3:1 hexane/acetone+1% TEA to 1:1 hexand/acetone+1% TEA. The product was obtained as a light yellow foam (9.454 g, 51% based upon recovered starting material). The recovery of the starting material was 28% (7.669 g). LCMS(ES) (MH⁺)=776.5.

To a solution of the above purified product (10 g, 12.9 mmol in 80 mL anhydrous EtOAc) at 0° C., 4-dimethylaminopyridine (DMAP, 6.36 g, 51.5 mmol), triethylamine (TEA, 7.21 mL, 51.5 mmol) and benzoic anhydride (11.89 g, 51.5 mmol) was added sequentially. The reaction was warmed to room temperature and stirred under argon for 20 hrs. TLC showed complete conversion of the starting material (5:1 toluene/acetone). The reaction was quenched by adding cold saturated aqueous NaHCO₃ and the organic layer was separated before extraction with dichloromethane. The organic layer was washed with brine, dried with Na₂SO₄, and concentrated in vacuo. Purification by column chromatography (silica gel, 8:1 hexane/acetone+1% TEA) yielded 11.23 g (89%) of the product. LCMS(ES) (MH⁺)=984.5.

A solution of the above purified material (8 g, 8.13 mmol in 80 mL dry EtOAc) was cooled to 0° C. under argon. Et₃N (4.6 mL, 32.5 mmol) was added quickly, before the fast dropwise addition of thionyl chloride (0.66 mL, 8.94 mmol). A white precipitate was formed. The reaction was kept at 0° C. for 1.5 hrs, at which time very little starting material remained, as shown by TLC. The reaction was quenched by adding cold saturated aqueous NaHCO₃ and the organic layer was separated. The aqueous layer was extracted with EtOAc. The organic layer was washed with brine, dried, and concentrated in vacuo. The crude material was purified by column chromatography (silica gel, 3:2 hexane/EtOAc+1% TEA) to afford Compound 13 (7.4 g, 94%). LCMS(ES) (MH⁺)=966.5.

Preparation of Compound 14

Step 1. To a 0° C. 0.1 M CH₂Cl₂ solution containing 13 was added mCPBA (5 eq). Warmed the reaction to rt and stirred for 16 h. Added cyclohexene (4 eq) and continued stirring for another 16 h. Poured into cold NaHCO₃ aq. and extracted with CH₂Cl₂ (3×). The organic extracts were washed with saturated NaHCO₃ aq. (6×) and brine (2×), dried with Na₂SO₄ and concentrated in vacuo to give N-oxide epoxide intermediate. This intermediate was dissolved in CH₂Cl₂ (0.1 M). To this solution at 0° C. was added sequentially isoproponol (2 eq) and tetra-n-propylammonium perruthenate (5 mol %). Warmed to rt and stirred for 16 h. Concentrated in vacuo to give a black residue. Purification by silica gel chromatography (5:1 hexane:acetone with 1% Et₃N) gave the epoxide product. ES/MS m/z 982.5 (MH⁺), C₅₄H₇₉NO₁₅=981.5 g/mol.

Step 2. A solution (0.1 M in anhydrous diethyl ether) of compound obtained from step 1 was added to dimethyl lithium cuprate (LiMe₂Cu) solution (0.1 M in anhydrous diethyl ether, 5 eq) at −78° C. The mixture was warmed to 0° C. and stirred under this temperature for 8 h. Poured into cold NH₄Cl aq. and the pH of aqueous was ˜7. Extracted with ether and CH₂Cl₂. The organic extracts were combined, washed with brine, dried over Na₂SO₄ and concentrated in vacuo. The resulting residue was purified by silica gel chromatography (5:1 hexane:acetone with 1% Et₃N) to give the C12-ethyl intermediate. ES/MS m/z 999 (MH⁺), C₅₅H₈₃NO₁₅=998 g/mol.

Step 3. An aqueous solution of acetic acid (100 eq) was added to acetonide from step 2 in MeCN to give a 0.08M MeCN:H₂O solution (2:1 v/v). The reaction was stirred for 70 h at 65-70° C. and neutralized with saturated NaHCO₃ aq. The reaction was extracted with CH₂Cl₂, and the organic layer was washed with brine, dried over Na₂SO₄, filtered, concentrated, and purified by silica gel chromatography (4:1 hexanes:acetone with 1% Et₃N) to afford 9,11-diol. ES/MS m/z 959 (MH⁺), C₅₂H₇₉NO₁₅=958 g/mol.

Step 4. To a 0° C. CH₂Cl₂ solution (0.2 M) of product obtained from step 3 was added tetra-n-propylammonium perruthenate (5 mol %), N-methylmorpholine N-oxide (1.2 equiv.) and 3 A molecular sieves (100 wt. %). The reaction was stirred under argon at 0° C. for 16 hrs. Diluted with EtOAc and filtered through a celite pad. The filtrate was concentrated in vacuo to give a residue which was purified by flash column chromatography (2:1 hexane/EtOAc+1% Et3N). Compound 14 (R=Me) was obtained as white foam. ES/MS m/z 956 (MH⁺), C₅₂H₇₇NO₁₅=955 g/mol.

### EXAMPLE 5

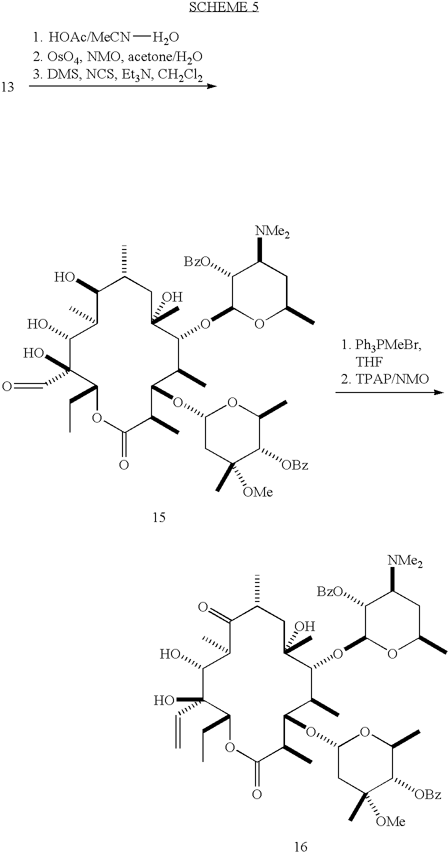 Preparation of Compound 15

Glacial acetic acid was added to a solution of Compound 13 (1 equiv) in MeCN/water (2:1, v/v) (0.08M). The reaction was stirred for 70 hrs at 65-70° C., cooled to ambient temperature, and neutralized with saturated aqueous NaHCO₃. The reaction mixture was extracted with methylene chloride and the organic extracts were washed sequentially with water and brine. The organic layer was dried over MgSO₄, filtered, and concentrated to yield a 9,11-diol intermediate product as a white solid. LCMS(ES) (MH⁺)=940.4.

To a solution of the 9,11-diol intermediate product obtained above (1 equiv in acetone/water (9:1, v/v)), N-methyl morpholine N-oxide mono-hydrate (2 equiv) was added before addition of osmium tetroxide in tert-butanol (0.08M). The solution was stirred at room temperature for 4 hrs. The solution was then diluted with ethyl acetate and cooled to 0° C. Upon cooling, saturated aqueous Na₂SO₃ was added and the solution was stirred for 10 min. The reaction was then warmed to room temperature, diluted with ethyl acetate, and washed with saturated aqueous NaHCO₃ and brine. The combined aqueous layers were back extracted with ethyl acetate, and the combined organic layers were dried with MgSO₄, filtered, and concentrated. Purification by column chromatography yielded a 9, 11, 12, 21-tetraol as a white solid. LCMS(ES) (MH⁺)=974.5.

To a solution of N-chlorosuccinimide (1 equiv) in dichloromethane at 0° C., methyl sulfide (1.2 equiv) is added. After stirring for 5 min, the solution is cooled to −20° C. To this solution, the above 9, 11, 12, 21-tetraol (0.9 equiv) in dichloromethane is added. The resulting solution is stirred at −23° C. for 95 min, at which time triethylamine (1 equiv) is added dropwise. After stirring at −20° C. for 5 min, the solution is allowed to warm to room temperature. The solution is then added to ethyl acetate, washed with saturated aqueous NaHCO₃ and brine. The organic layer is dried with MgSO₄, filtered, and concentrated. The crude material is purified by column chromatography (silica gel, MeOH/CH₂Cl₂/NH₄OH or hexanes/acetone/TEA) to yield Compound 15.

Preparation of Compound 16

To a solution of methyl triphenylphosphonium bromide (2 equiv) in tetrahydrofuran at −78° C., potassium bis(trimethylsilyl)amide (0.5M in toluene, 2 equiv) is added. The cooling bath is then removed and the anion solution is stirred for 1 hr before cooling the solution back to −78° C. The aldehyde Compound 15 (0.5 equiv) in tetrahydrofuran is added. The cooling bath is removed and the anion solution is stirred for 4-8 hrs, at which time ethyl acetate and saturated aqueous NH₄Cl are added. After the formation of two layers, the reaction is added to ethyl acetate and saturated aqueous NH₄Cl. The organic layer is separated, washed with saturated aqueous NaHCO₃ and brine, dried with MgSO₄, filtered, and concentrated. The crude material is purified by column chromatography (silica gel, MeOH/CH₂Cl₂/NH₄OH or hexanes/acetone/TEA) to yield a 12,21-ene macrolide product.

To a CH₂Cl₂ solution of the 12,21-ene macrolide product (0.2M) obtained from above, tetra-n-propylammonium perruthenate (5 mol %), N-methylmorpholine N-oxide (1.2 equiv.) and 3 Å molecular sieves (100 wt. %) is added at 0° C. The reaction is stirred under argon at 0° C. for 16 hrs. The reaction is diluted with EtOAc and filtered through a Celite pad. The filtrate is concentrated in vacuo to give a residue. The crude material is purified by column chromatography (silica gel, MeOH/CH₂Cl₂/NH₄OH or hexanes/acetone/TEA) to yield C₁₂-vinyl macrolide Compound 16.

### EXAMPLE 6

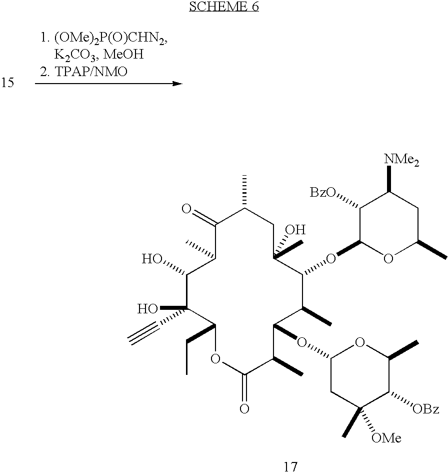 Preparation Compound 17

Compound 15 (1 equiv) is added to a 0° C. solution of Seyferth-Gilbert reagent, dimethyl diazomethylphosphonate, (1.20 eq, synthesized according to references: J. Org. Chem. 1996, Vol. 61, p. 2540 and 1971, Vol. 36, p. 1379, the disclosures of which are incorporated herein by reference) in dry MeOH. Potassium carbonate (2.00 equiv) is added. The solution is stirred at 0° C. for 1.5 hours and then brought up to ambient temperature for approximately 1-4 hrs. The reaction mixture is poured into ethyl acetate and washed sequentially with water and brine. The organic layer is dried over MgSO₄, filtered, and concentrated. Column chromatography (silica gel, MeOH/CH₂Cl₂/NH₄OH or hexanes/acetone/TEA) yields a 12-acetylene product.

The above 12-acetylene product is dissolved into a CH₂Cl₂ solution (0.2M) at 0° C. before addition of tetra-n-propylammonium perruthenate (5 mol %), N-methylmorpholine N-oxide (1.2 equiv), and 3 A molecular sieves (100 wt. %). The reaction is stirred under argon at 0° C. for 16 hrs. The reaction is diluted with EtOAc and filtered through a Celite pad. The filtrate is concentrated in vacuo to give a residue. The crude material is purified by column chromatography (silica gel, MeOH/CH₂Cl₂/NH₄OH or hexanes/acetone/TEA) to yield the 12-acetylene macrolide Compound 17.

### EXAMPLE 7

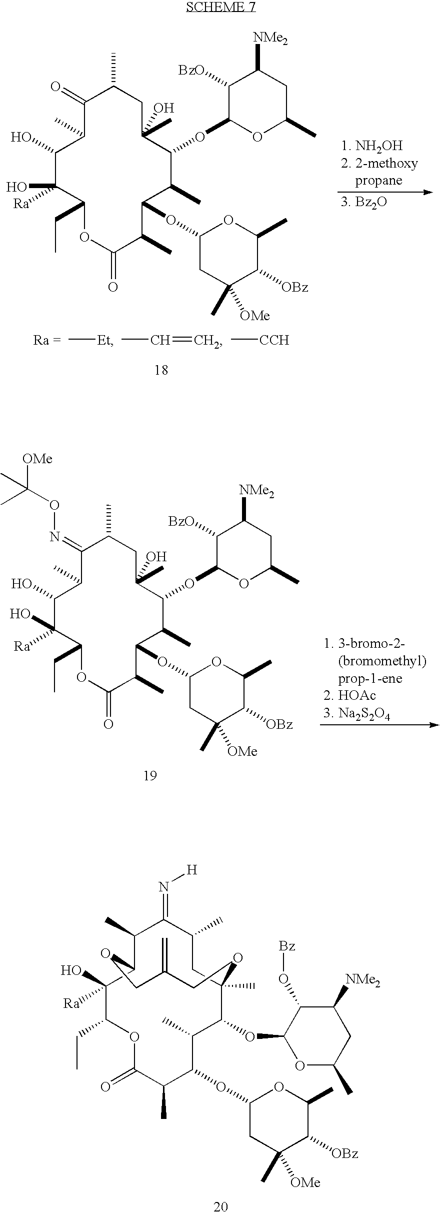 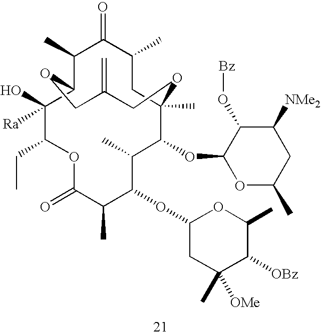 Preparation of Compound 19

Oxime formation. A 50% (w/w) aqueous solution of hydroxylamine (13 equiv) is added to a 0.5M solution of Compound 18 in 2-propanol. Glacial acetic acid (4.2 equiv) is added. The mixture is stirred at 50° C. for 18 hrs and then returned to ambient temperature. The reaction mixture is poured into dichloromethane and saturated aqueous sodium bicarbonate. The pH of the aqueous layer is adjusted to 9 with 6N sodium hydroxide, and the layers are separated. The organic phase is washed with brine, dried over magnesium sulfate, filtered, and concentrated. The crude material is purified by flash chromatography over silica gel (2:1 hexanes:acetone+2% triethylamine) to give an intermediate oxime product.

Oxime protection. A 0.3M solution of the above intermediate oxime product in dichloromethane is cooled to 0° C. 2,2-dimethoxypropane (10 equiv) and pyridinium p-toluenesulfonate (2 equiv) are added. After 0.5 hrs, the reaction is brought to ambient temperature. The mixture is stirred for 48 hrs and poured into dichloromethane and saturated aqueous sodium bicarbonate. The layers are separated. The organic phase is washed with water then brine, dried over magnesium sulfate, filtered, and concentrated. The crude material is re-dissolved in toluene and concentrated. The material may be used without further purification.

2′-O-benzoylation. Benzoic anhydride (2 equiv) is added to a 0.2M solution of the above oxime protected crude material in EtOAc. The mixture is stirred at ambient temperature for 8 hrs and then poured into EtOAc and saturated sodium bicarbonate. The layers are separated. The organic layer is washed with water and brine, dried over magnesium sulfate, filtered, and concentrated. The crude material is purified by flash chromatography over silica gel to give Compound 19.

Preparation of Compounds 20 and 21

6,11-bis-O-alkylation. A 0.1M solution of Compound 19 in 5:5:1 THF:DMSO:ether is cooled to 0° C. Freshly distilled 3-bromo-2-(bromomethyl)prop-1-ene (4 equiv) is added. A 0.5M solution of potassium tert-butoxide in 1:1 THF:DMSO (3 equiv) is added over 2 hrs while keeping the reaction mixture at 5-10° C. The mixture is poured into EtOAc and saturated sodium bicarbonate. The layers are separated. The organic layer is washed with water and brine, dried over magnesium sulfate, filtered, and concentrated. The crude material is used without further purification.

Alternatively, compounds 20 and 21 may be prepared via 19a using the methyl or t-Butyl dicarbonate.

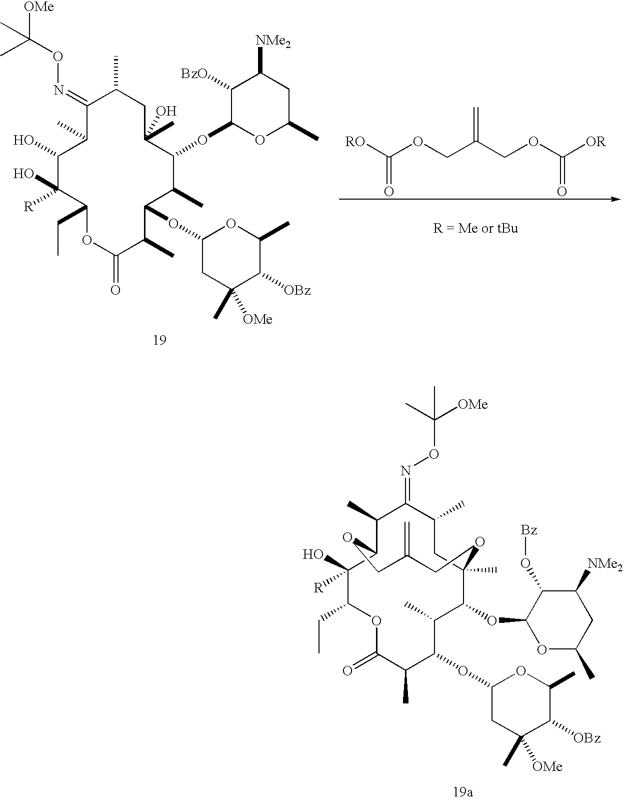

An anhydrous and air free system of dilute solution (0.001˜0.05 M) containing compound 19, dicarbonate A (can be prepared from 2-methylene-1,3-propanediol), tetra-kis-triphenylphosphine palladium(0) or palladium(II) acetate with ligands such as triphenylphosphine and (oTolyl)₃P or Pd₂(dba)₃ and dppb in THF is heated slowly to reflux for 0.5 h to 12 hrs. The reaction is then cooled to room temperature and concentrated in vacuo. The resulting residue is purified by chromatography on a silica gel column to provide 6,11-bis-alkylated compound as 19a.

Deprotection. A 0.1M solution of the above crude material in 2:1:1 acetonitrile:water:HOAc is stirred overnight at ambient temperature. Toluene and 2-propanol are added, and the mixture is concentrated under reduced pressure. The residue is re-dissolved in toluene and concentrated under reduced pressure. The crude material is used without further purification.

9-Imine/9-Keto. A 0.1 M solution of the above crude material in 1:1 EtOH:water is treated with sodium hydrosulfite (5.5 equiv) and formic acid (4.7 equiv). The mixture is stirred at 80° C. for 5 hrs and then returned to ambient temperature. The reaction is quenched by addition of sodium bicarbonate and extracted with EtOAc. The combined extracts are washed sequentially with sodium bicarbonate, water, and brine. The organic layer is dried over magnesium sulfate, filtered, and concentrated. The crude material (mixture of 20 and 21) is purified by flash chromatography over silica gel to give 9-imine Compound 20 and 9-keto Compound 21.

### EXAMPLE 8

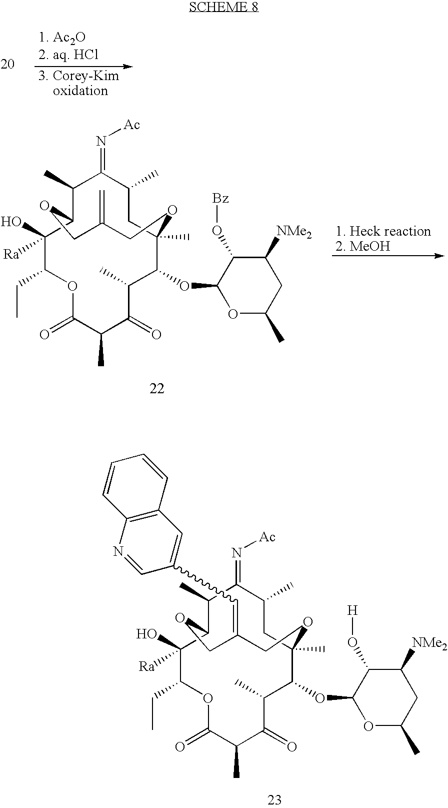 Preparation of Compound 22

Acylation of 9-imine. Acetic anhydride (2 equiv) is added to a 0.2M solution of Compound 20 in EtOAc. The mixture is stirred at ambient temperature for 8 hrs and then poured into EtOAc and saturated sodium bicarbonate. The layers are separated. The organic layer is washed with water and brine, dried over magnesium sulfate, filtered, and concentrated. The crude material is purified by flash chromatography over silica gel to give a 9-acetylimine intermediate product.

Removal of cladinose. A 0.075M solution of the above 9-acetylimine intermediate product in 1:1 acetonitrile:3N aqueous HCl is stirred overnight at ambient temperature. The mixture is cooled to 0° C. and neutralized with 6N aqueous sodium hydroxide. Volatiles are removed under reduced pressure, and the resulting syrup is extracted with EtOAc. The combined extracts are washed sequentially with sodium bicarbonate, water, and brine. The organic layer is dried over magnesium sulfate, filtered, and concentrated. The crude material is purified by flash chromatography over silica gel to give an intermediate compound.

Oxidation of 3-OH. Methyl sulfide (1.75 equiv) is added to a 0.1M solution of N-chlorosuccinimide (1.5 equiv) in dichloromethane at −10° C. The mixture is stirred for 15 min. A 0.1M solution of the above intermediate compound (1.0 equiv) in dichloromethane is added dropwise over 10 min. The mixture is stirred an additional 30 min and then quenched with triethylamine (1.0 equiv). The reaction is brought to 0° C. over 30 min and then poured into EtOAc and saturated sodium bicarbonate. The layers are separated. The organic layer is washed with water and brine, dried over magnesium sulfate, filtered, and concentrated. The crude material is purified by flash chromatography over silica gel to give Compound 22.

Preparation of Compound 23

Heck reaction. Tris(dibenzylideneacetone)dipalladium(0) chloroform adduct (0.25 equiv) is added to a degassed 0.1 M solution of Compound 22, tri-O-tolylphosphine (1.0 equiv), 3-bromoquinoline (10 equiv), and triethylamine (2.0 equiv) in acetonitrile. The mixture is stirred at 70° C. for 30 hrs and returned to ambient temperature. The reaction mixture is poured into EtOAc and saturated sodium bicarbonate. The layers are separated. The organic layer is washed with water and brine, dried over magnesium sulfate, filtered through Celite, and concentrated. The crude material is purified by flash chromatography over silica gel to give the intermediate compound.

Deprotection. A 0.05M solution of the above intermediate compound is stirred in methanol at 70° C. for 16 hrs. The mixture is returned to ambient temperature, and volatiles are removed under reduced pressure. Purification by flash chromatography over silica gel gives Compound 23.

### EXAMPLE 9

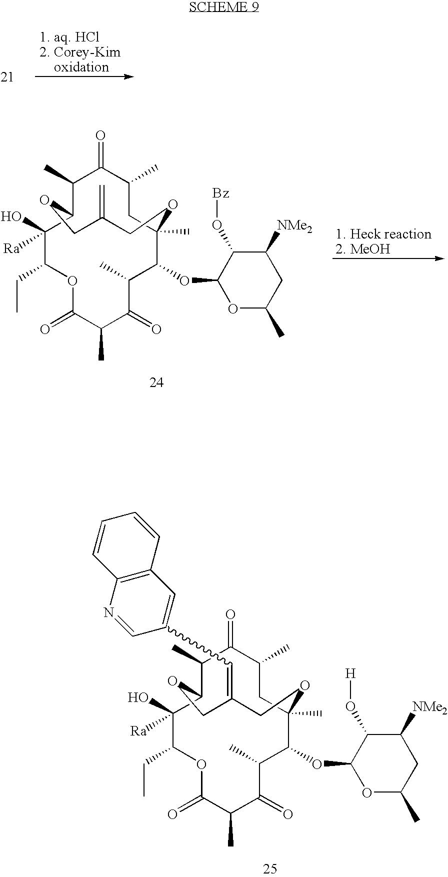 Preparation of Compound 24

Removal of cladinose. A 0.075M solution of Compound 21 in 1:1 acetonitrile:3N aqueous HCl is stirred overnight at ambient temperature. The mixture is cooled to 0° C. and neutralized with 6N aqueous sodium hydroxide. Volatiles are removed under reduced pressure, and the resulting syrup is extracted with EtOAc. The combined extracts are washed sequentially with sodium bicarbonate, water, and brine. The organic layer is dried over magnesium sulfate, filtered, and concentrated. The crude material is purified by flash chromatography over silica gel to give an intermediate compound.

Oxidation of 3-OH. Methyl sulfide (1.75 equiv) is added to a 0.1M solution of N-chlorosuccinimide (1.5 equiv) in dichloromethane at −10° C. The mixture is stirred for 15 min. A 0.1M solution of the above intermediate compound (1.0 equiv) in dichloromethane is added dropwise over 10 min. The mixture is stirred an additional 30 min and then quenched with triethylamine (1.0 equiv). The reaction is brought to 0° C. over 30 min and then poured into EtOAc and saturated sodium bicarbonate. The layers are separated. The organic layer is washed with water and brine, dried over magnesium sulfate, filtered, and concentrated. The crude material is purified by flash chromatography over silica gel to give Compound 24.

Preparation of Compound 25

Heck reaction. Tris(dibenzylideneacetone)dipalladium(0) chloroform adduct (0.25 equiv) is added to a degassed 0.1 M solution of Compound 24, tri-O-tolylphosphine (1.0 equiv), 3-bromoquinoline (10 equiv), and triethylamine (2.0 equiv) in acetonitrile. The mixture is stirred at 70° C. for 30 hrs and returned to ambient temperature. The reaction mixture is poured into EtOAc and saturated sodium bicarbonate. The layers are separated. The organic layer is washed with water and brine, dried over magnesium sulfate, filtered through Celite, and concentrated. The crude material is purified by flash chromatography over silica gel to give an intermediate compound.

Deprotection. A 0.05M solution of the above intermediate compound is stirred in methanol at 70° C. for 16 hrs. The mixture is returned to ambient temperature, and volatiles are removed under reduced pressure. Purification by flash chromatography over silica gel gives Compound 25.

### EXAMPLE 10

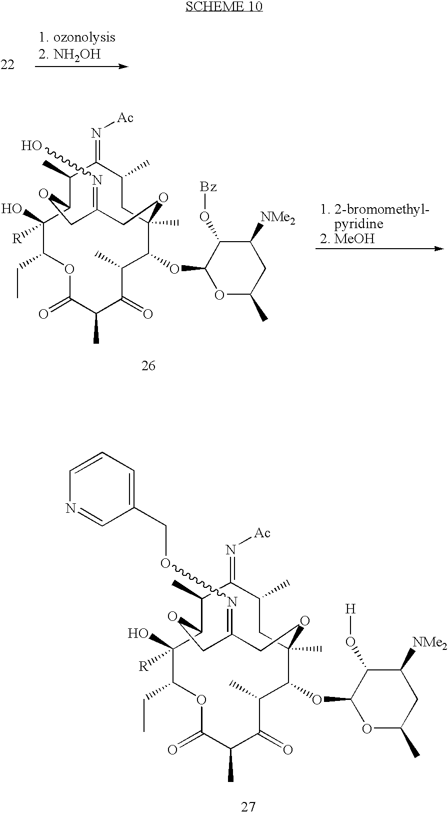 Preparation of Compound 26

Ozonolysis. To a mixture of olefin Compound 22 and toluenesulfonic acid monohydrate (1.2 equiv) in dichloromethane/methanol (19:1 v/v, 0.02M) at −78° C., ozone gas is bubbled in until a medium blue color appeared. The reaction is stirred for an additional ten minutes and purged with nitrogen until the solution becomes colorless. After adding dimethyl sulfide (3 equiv), the solution is stirred for ten minutes, treated with triethylamine (5 equiv), warmed to room temperature and concentrated. Purification by silica gel chromatography affords a ketone intermediate product.

Oxime formation. A 50% (w/w) aqueous solution of hydroxylamine (13 equiv) is added to a 0.5M solution of the above ketone intermediate product in 2-propanol. Glacial acetic acid (4.2 eq) is added. The mixture is stirred at between 25° C. to 50° C. for 18 hrs. The reaction mixture is poured into dichloromethane and saturated aqueous sodium bicarbonate. The pH of the aqueous layer is adjusted to 9 with 6N sodium hydroxide, and the layers are separated. The organic phase is washed with brine, dried over magnesium sulfate, filtered, and concentrated. The crude material is purified by flash chromatography over silica to give Compound 26.

Preparation of Compound 27

Allylation. A mixture of Compound 26, potassium carbonate (1.5 equiv) and 2-bromomethyl-pyridine (1.2 equiv) in DMF is stirred between 25° C. to 50° C. for approximately 2-16 hrs. The reaction is diluted with water and extracted with dichloromethane. The organic extracts are washed with brine, dried over magnesium sulfate, filtered, and concentrated. The crude material is purified by flash chromatography over silica to give an intermediate allylation product.

Deprotection. A 0.05M solution of the above intermediate allylation product is stirred in methanol at 70° C. for 16 hrs. The mixture is returned to ambient temperature, and volatiles are removed under reduced pressure. Purification by flash chromatography over silica gel gives Compound 27.

### EXAMPLE 11

Compounds are assayed in vitro for antibacterial activity according to the following procedure:

Strains. The bacterial isolates are cultivated from −70° C. frozen stocks by two consecutive overnight passages (P1, P2) at 35° C. on 5% blood agar (Remel, Lenexa, Kans.). Chocolate agar (Remel) is used for Haemophilus influenzae. H. influenzae and Streptococcus pneumoniae are incubated in 5-10% CO₂.

Drug Stock Preparation. To determine the amount of solvent to be used to give the desired final concentration, the formula “weight obtained in mg/final concentration in mg/ml” will be used. It will give the amount of solvent in mL needed to be added to give the desired concentration. For example, if 2.5 mg/mL is the desired concentration and the weight of compound is 13.7 mg, then the amount of solvent to be added is 3.94 ml (13.7 mg/2.5 mg/ml=3.94 mL). Methanol is used as the solvent to dissolve the test compounds. Further dilution of stock is done in sterile, deioinzed water. Drug stocks are kept frozen at −70° C., protected from light.

Susceptibility Testing. MICs are determined by the broth microdilution method in accordance with the NCCLS guidelines. In brief, organism suspensions are adjusted to a 0.5 McFarland standard to yield a final inoculum between 3×10⁵ and 7×10⁵ CFU/ml. Drug dilutions and inocula are made in sterile, cation adjusted Mueller-Hinton Broth (CAMHB) (Remel) for all but S. pneumoniae [CAMHB with 2-5% lysed horse blood (Remel)] and H. influenzae [Haemophilus Test Medium (Remel)]. An inoculum volume of 100 μl is added to wells containing 100 μl of broth with 2-fold serial dilutions of drug. All inoculated microdilution trays are incubated in ambient air at 35° C. for 18-24 hours, except for S. pneumoniae, and H. influenzae (both at 5-10% CO₂).

Following appropriate incubation, the MIC is determined and the MIC is defined as the lowest concentration of the drug that prevented visible growth.

While the preferred embodiments of the invention have been illustrated and described, it will be appreciated that various changes can be made therein without departing from the spirit and scope of the invention.

1. A compound having the formula (Q): 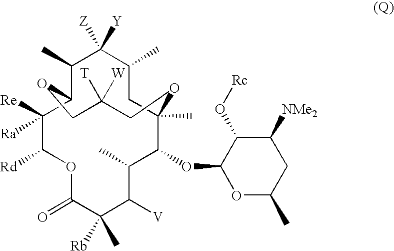
or a stereoisomer, tautomer, pharmaceutically acceptable salt, ester or prodrug thereof, wherein
V is —OCORx, carbonyl, or a cladinose moiety of the formula: 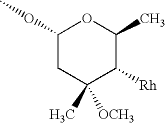
wherein Rx is H, alkyl, —O-alkyl, —N(H)-alkyl, or —N(alkyl)₂;
either Y and Z taken together define a group X, wherein X is selected from the group consisting of
(1) ═O,
(2) ═N—OH,
(3) ═N—O—R¹, wherein R¹ is selected from the group consisting of
(a) C₁-C₁₂-alkyl,
(b) C₁-C₁₂-alkyl substituted with alkoxy,
(c) C₁-C₁₂-alkyl substituted with aryl,
(d) C₁-C₁₂-alkyl substituted with substituted aryl,
(e) C₁-C₁₂-alkyl substituted with heteroaryl,
(f) C₁-C₁₂-alkyl substituted with substituted heteroaryl,
(g) C₃-C₁₂-cycloalkyl, and
(h) —Si—(R²)(R³)(R⁴), wherein R², R³, and R⁴ are each independently selected from C₁-C₁₂-alkyl and aryl;
(4) ═N—O—C(R⁵)(R⁶)—O—R¹, wherein R¹ is as previously defined and R⁵ and R⁶ are each independently selected from the group consisting of
(a) hydrogen,
(b) C₁-C₁₂-alkyl,
(c) C₁-C₁₂-alkyl substituted with aryl,
(d) C₁-C₁₂-alkyl substituted with substituted aryl,
(e) C₁-C₁₂-alkyl substituted with heteroaryl, and
(f) C₁-C₁₂-alkyl substituted with substituted heteroaryl,
or R⁵ and R⁶ taken together with the atoms to which they are attached form a C₃-C₁₂-cycloalkyl ring;
(5) ═N—C(O)R¹⁵, wherein R¹⁵ is selected from the group consisting of
(a) C₁-C₁₂-alkyl,
(b) substituted C₁-C₁₂-alkyl,
(c) C₂-C₁₂-alkenyl,
(d) substituted C₂-C₁₂-alkenyl,
(e) C₂-C₁₂-alkynyl,
(f) substituted C₂-C₁₂-alkynyl,
(g) aryl,
(h) C₃-C₈-cycloalkyl,
(i) substituted C₃-C₈-cycloalkyl,
(j) substituted aryl,
(k) heterocycloalkyl,
(l) substituted heterocycloalkyl,
(m) C₁-C₁₂-alkyl substituted with aryl,
(n) C₁-C₁₂-alkyl substituted with substituted aryl,
(o) C₁-C₁₂-alkyl substituted with heterocycloalkyl,
(p) C₁-C₁₂-alkyl substituted with substituted heterocycloalkyl,
(q) C₁-C₁₂-alkyl substituted with C₃-C₈-cycloalkyl,
(r) C₁-C₁₂-alkyl substituted with substituted C₃-C₈-cycloalkyl,
(s) heteroaryl,
(t) substituted heteroaryl,
(u) C₁-C₁₂-alkyl substituted with heteroaryl, and
(v) C₁-C₁₂-alkyl substituted with substituted heteroaryl;
(w) C₁-C₁₂-alkyl substituted with alkylalkoxy,
(x) C₁-C₆-alkoxy,
(y) C₁-C₆-thioalkoxy,
(z) amino,
(aa) alkylamino,
(bb) dialkylamino,
(cc) —NO₂, and
(dd) —COOH,
or one of Y and Z is hydrogen and the other is selected from a group consisting of
(1) hydroxyl,
(2) protected hydroxyl, and
(3) —NR⁷R⁸, wherein R⁷ and R⁸ are independently selected from hydrogen and alkyl, substituted alkyl, or R⁷ and R⁸ are taken with the nitrogen atom to which they are connected to form a 3- to 7-membered ring which, when the ring is a 5- to 7-membered ring, may optionally contain a hetero function selected from the group consisting of —O—, —NH, —N(C₁-C₆-alkyl)-, —N(aryl)-, —N(aryl-C₁-C₆-alkyl-)-, —N(substituted-aryl-C₁-C₆-alkyl-)-, —N(heteroaryl)-, —N(heteroaryl-C₁-C₆-alkyl-)-, —N(substituted-heteroaryl-C₁-C₆-alkyl-)-, and —S— or —S(O)ₙ—, wherein n is 1 or 2;
Ra is selected from the group consisting of
(1) hydrogen;
(2) C₁ alkyl further substituted with one or more substituents selected from a group consisting of
(a) hydroxyl,
(b) halogen,
(c) thiol, which can be further substituted with an C₁-C₁₂-alkyl or substituted C₁-C₁₂-alkyl group,
(d) C₁-C₁₂-alkyl, which can be further substituted by halogen, hydroxyl, C₁-C₁₂-alkoxy, or amino,
(e) C₁-C₃-alkoxy,
(f) C₁-C₃-thioalkoxy,
(g) amino,
(h) C₁-C₁₂-alkylamino,
(i) C₁-C₁₂-dialkylamino,
(j) —CN,
(k) —NO₂,
(l) —CONH₂,
(m) —COOH,
(n) —CO₂R¹⁰, wherein R¹⁰ is C₁-C₃-alkyl, aryl substituted with C₁-C₃-alkyl, or heteroaryl substituted with C₁-C₃-alkyl,
(o)—N₃;
(3) C₂-C₁₂-alkyl;
(4) substituted C₁-C₁₂-alkyl;
(5) C₂-C₄-alkenyl, which can be further substituted with C₁-C₁₂-alkyl and one or more halogen groups;
(6) C₂-C₄-alkynyl, which can be further substituted with C₁-C₁₂-alkyl and one or more halogen groups;
(7) aryl, which can be further substituted with C₁-C₁₂-alkyl and one or more halogen groups;
(8) —CHO;
(9) —CO₂H;
(10) —CN;
(11) —CO₂R¹⁰, wherein R¹⁰ is as previously defined;
(12) —C(O)NR¹¹R¹², wherein R¹¹ and R¹² are independently selected from hydrogen, C₁-C₃-alkyl, C₁-C₃-alkyl substituted with aryl, substituted aryl, heteroaryl, and substituted heteroaryl;
(13) —C(O)R⁹, wherein R⁹ is selected from the group consisting of
(a) alkyl optionally substituted with a substituent selected from the group consisting of
(k) aryl,
(ii) substituted aryl,
(iii)heteroaryl, and
(iv) substituted heteroaryl,
(b) aryl,
(c) substituted aryl,
(d) heteroaryl,
(e) substituted heteroaryl, and
(f) heterocycloalkyl, and
(14) thioester;
Rb is hydrogen, halogen, or C₁-C₁₂-alkyl which can be further substituted by one or more halo groups, or Rb can be taken together with V to form a double bond;
Rc is hydrogen or a hydroxyl protecting group;
Rd is selected from the group consisting of
(1) C₁-C₁₂-alkyl,
(2) C₁-C₁₂-alkyl substituted with one or more substituents selected from the group consisting of
(a) halogen,
(b) hydroxyl, and
(c) C₁-C₃-alkoxy,
(3) C₃-C₇-cycloalkyl,
(4) C₂-C₄-alkenyl, and
(5) C₂-C₄-alkynyl;
Re is hydroxyl, amino, or alkylamino; or Re and Ra may be taken together to form an epoxide, a carbonyl, an olefin, or a substituted olefin; or Re and Ra when taken together with the atom to which they are attached form a spiro ring consisting of C₃-C₇-carbocyclic, carbonate, or carbamate, wherein the nitrogen atom can be unsubstituted or substituted with an alkyl group;
Rh is selected from the group consisting of
(1) hydrogen,
(2) —ORj, wherein Rj is hydrogen or a hydroxyl protecting group,
(3) halogen, and
(4) —OC(O)NHRi, wherein Ri is selected from a group consisting of
(a) C₁-C₄ alkyl,
(b) C₁-C₄ aminoalkyl where the amino group is substituted with one or two groups selected from
(i) C₁-C₄ alkyl,
(ii) C₁-C₄ alkyl substituted with halogen,
(iii) C₁-C₄ alkyl substituted with alkoxy,
(iv) C₁-C₄ alkyl substituted with hydroxyl,
(v) C₁-C₄ alkyl substituted with aryl,
(vi) C₁-C₄ alkyl substituted with substituted aryl,
(vii) C₁-C₄ alkyl substituted with heteroaryl,
(viii) C₁-C₄ alkyl substituted with substituted heteroaryl, and
(ix) C₃-C₆ cycloalkyl;
T and W taken together define a group R, wherein R is selected from the group consisting of
(1) ═O;
(2) ═CH(Rk), wherein Rk is a cis or trans substituent selected from the group consisting of
(a) hydrogen,
(b) halogen selected from the group consisting of Br, Cl, F, and I,
(c) C₁-C₁₂-alkyl,
(d) C₁-C₁₂-alkyl substituted with aryl,
(e) C₁-C₁₂-alkyl substituted with substituted aryl,
(f) C₁-C₁₂-alkyl substituted with heteroaryl,
(g) C₁-C₁₂-alkyl substituted with substituted heteroaryl,
(h) C₂-C₁₂-alkenyl,
(i) C₂-C₁₂-alkenyl substituted with aryl,
(j) C₂-C₁₂-alkenyl substituted with substituted aryl,
(k) C₂-C₁₂-alkenyl substituted with heteroaryl,
(l) C₂-C₁₂-alkenyl substituted with substituted heteroaryl,
(m) C₂-C₁₂-alkynyl,
(n) C₂-C₁₂-alkynyl substituted with aryl,
(o) C₂-C₁₂-alkynyl substituted with substituted aryl,
(p) C₂-C₁₂-alkynyl substituted with heteroaryl,
(q) C₂-C₁₂-alkynyl substituted with substituted heteroaryl,
(r) aryl,
(s) substituted aryl,
(t) heteroaryl,
(u) substituted heteroaryl; and
(3) ═N—O-(Rm), wherein Rm is selected from the group consisting of
(a) hydrogen,
(b) C₁-C₁₂-alkyl,
(c) C₁-C₁₂-alkyl substituted with C₁-C₁₂-alkenyl,
(d) C₁-C₁₂-alkyl substituted with substituted C₁-C₁₂-alkenyl,
(e) C₁-C₁₂-alkyl substituted with aryl,
(f) C₁-C₁₂-alkyl substituted with substituted aryl,
(g) C₁-C₁₂-alkyl substituted with heteroaryl,
(h) C₁-C₁₂-alkyl substituted with substituted heteroaryl,
(i) C₂-C₁₂-alkenyl,
(j) C₂-C₁₂-alkenyl substituted with aryl,
(k) C₂-C₁₂-alkenyl substituted with substituted aryl,
(l) C₂-C₁₂-alkenyl substituted with heteroaryl,
(m) C₂-C₁₂-alkenyl substituted with substituted heteroaryl,
(n) aryl,
(o) substituted aryl,
(p) heteroaryl, and
(q) substituted heteroaryl.

2. The compound of claim 1, wherein
V is carbonyl;
Y and Z are taken together define a group X, wherein X is ═N—C(O)R¹⁵, wherein R¹⁵ is selected from the group consisting of
(1) C₁-C₁₂-alkyl,
(2) substituted C₁-C₁₂-alkyl,
(3) C₁-C₆-alkoxy, and
(4) amino;
Ra is selected from the group consisting of hydrogen, C₂-C₁₂-alkyl, substituted C₁-C₁₂-alkyl, C₂-C₄-alkenyl, and C₂-C₄-alkynyl;
Rb is hydrogen or halogen;
Rc is hydrogen or —C(O)CH₃;
Rd is ethyl;
Re is hydroxyl; and
R is selected from the group consisting of
(1) ═O;
(2) ═CH(Rk), wherein Rk is a cis or trans substituent selected from the group consisting of
(a) hydrogen,
(b) halogen, wherein said halogen is Br,
(c) C₂-C₁₂-alkenyl substituted with aryl,
(d) C₂-C₁₂-alkenyl substituted with substituted aryl,
(e) C₂-C₁₂-alkenyl substituted with heteroaryl,
(f) C₂-C₁₂-alkynyl substituted with aryl,
(g) C₂-C₁₂-alkynyl substituted with heteroaryl,
(h) C₂-C₁₂-alkynyl substituted with substituted heteroaryl,
(i) aryl,
(j) substituted aryl, and
(k) heteroaryl; and
(3) ═N—O-(Rm), wherein Rm is selected from the group consisting of
(a) C₁-C₁₂-alkyl substituted with substituted C₁-C₁₂-alkenyl,
(b) C₁-C₁₂-alkyl substituted with aryl,
(c) C₁-C₁₂-alkyl substituted with substituted aryl,
(d) C₁-C₁₂-alkyl substituted with heteroaryl,
(e) C₁-C₁₂-alkyl substituted with substituted heteroaryl, and
(f) aryl.

3. The compound of claim 2, wherein
V is carbonyl;
Y and Z are taken together define a group X, wherein X is ═N—C(O)R¹⁵, wherein R¹⁵ is selected from the group consisting of
(1) C₁-C₁₂-alkyl, wherein said C₁-C₁₂-alkyl is methyl or ethyl;
(2) substituted C₁-C₁₂-alkyl, wherein said substituted C₁-C₁₂-alkyl is —CH₂OH, —CH₂OC(O)CH₃, —CH₂OCH₃, or —CH₂NH₂;
(3) C₁-C₆-alkoxy, wherein said C₁-C₆-alkoxy is —OCH₃; and
(4) amino;
Ra is selected from the group consisting of hydrogen, C₂-C₁₂-alkyl, substituted C₁-C₁₂-alkyl, C₂-C₄-alkenyl, and C₂-C₄-alkynyl;
Rb is hydrogen or fluorine;
Rc is hydrogen or —C(O)CH₃;
Rd is ethyl;
Re is hydroxyl; and
R is selected from the group consisting of
(1) ═O;
(2) ═CH(Rk), wherein Rk is a cis or trans substituent selected from the group consisting of
(a) hydrogen,
(b) halogen, wherein said halogen is Br,
(c) C₂-C₁₂-alkenyl substituted with aryl, wherein said C₂-C₁₂-alkenyl substituted with aryl is —CH═CH(C₆H₅),
(d) C₂-C₁₂-alkenyl substituted with substituted aryl, wherein said C₂-C₁₂-alkenyl substituted with substituted aryl is 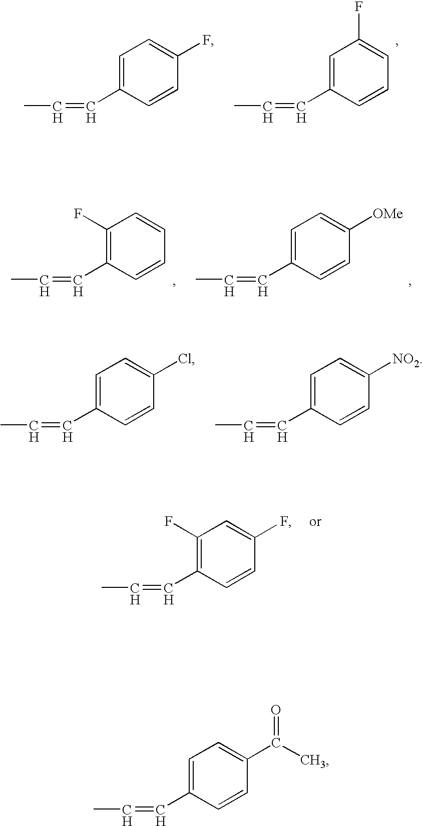
(e) C₂-C₁₂-alkenyl substituted with heteroaryl, wherein said C₂-C₁₂-alkenyl substituted with heteroaryl is 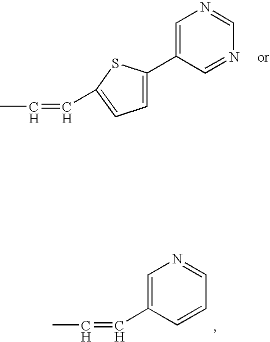
(f) C₂-C₁₂-alkynyl substituted with aryl, wherein said C₂-C₁₂-alkynyl substituted with aryl is 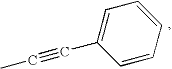
(g) C₂-C₁₂-alkynyl substituted with heteroaryl, wherein said C₂C₁₂-alkynyl substituted with heteroaryl is 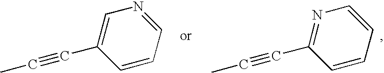
(h) C₂-C₁₂-alkynyl substituted with substituted heteroaryl, wherein said C₂-C₁₂-alkynyl substituted with substituted heteroaryl is 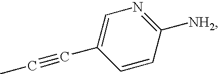
(i) aryl, wherein said aryl is —C₆H₅, 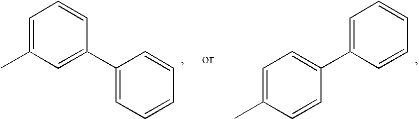
(j) substituted aryl, wherein said substituted aryl is 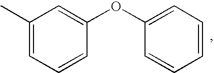
(k) heteroaryl, wherein said heteroaryl is or 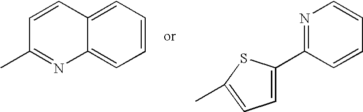
; and
(3) ═N—O-(Rm), wherein Rm is selected from the group consisting of
(a) C₁-C₁₂-alkyl substituted with substituted C₁-C₁₂-alkenyl, wherein said C₁-C₁₂-alkyl substituted with substituted C₁-C₁₂-alkenyl is —CH₂—CH═CH(C₆H₅), (b) C₁-C₁₂-alkyl substituted with aryl, wherein said C₁-C₁₂-alkyl substituted with aryl is —CH₂(C₆H₅), —CH₂CH₂(C₆H₅), or
—CH₂CH₂CH₂(C₆H₅),
(c) C₁-C₁₂-alkyl substituted with substituted aryl, wherein said C₁-C₁₂-alkyl substituted with substituted aryl is 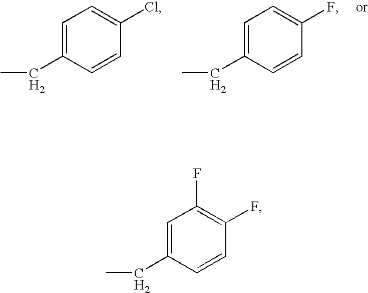
(d) C₁-C₁₂-alkyl substituted with heteroaryl, wherein said C₁-C₁₂-alkyl substituted with heteroaryl is 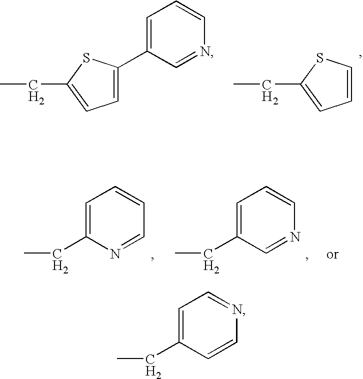
(e) C₁-C₁₂-alkyl substituted with substituted heteroaryl, wherein said C₁-C₁₂-alkyl substituted with substituted heteroaryl is 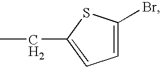
and
(f) aryl, wherein said aryl is —C₆H₅.

4. The compound of claim 1, wherein Ra is selected from the group consisting of hydrogen, C₂-C₁₂-alkyl, substituted C₁-C₁₂-alkyl, C₂-C₄-alkenyl, and C₂-C₄-alkynyl.

5. The compound of claim 4, wherein said C₂-C₁₂-alkyl group is ethyl.

6. The compound of claim 4, wherein said C₂-C₄-alkenyl group is vinyl.

7. The compound of claim 4, wherein said C₂-C₄-alkynyl group is acetylene.

8. The compound of claim 1 having the formula 693:

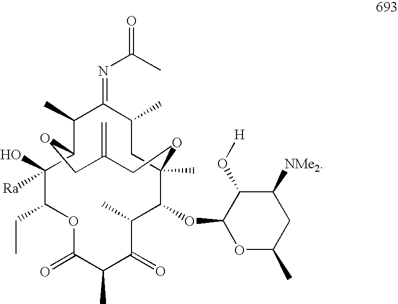

9. The compound of claim 1 having the formula 308:

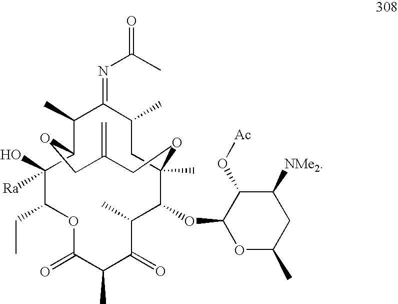

10. The compound of claim 1 having formula 218:

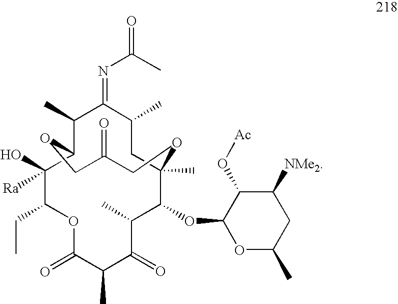

11. The compound of claim 1 having formula 589:

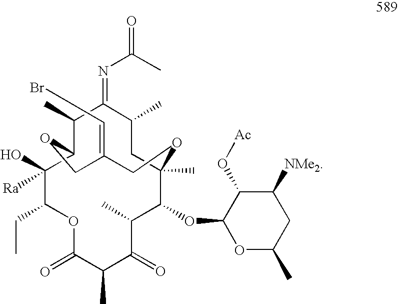

12. The compound of claim 1 having formula 879:

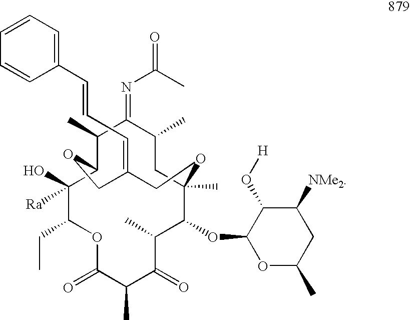

13. The compound of claim 1 having formula 400:

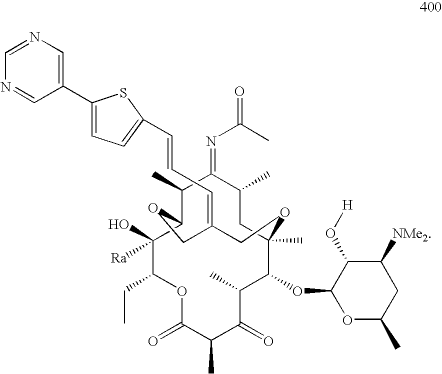

14. The compound of claim 1 having formula 539:

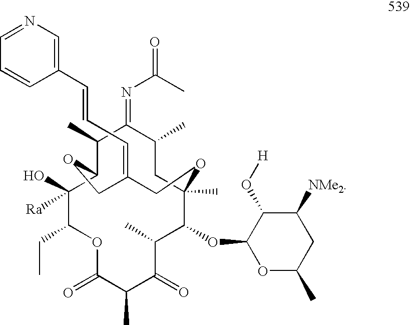

15. The compound of claim 1 having formula 643:

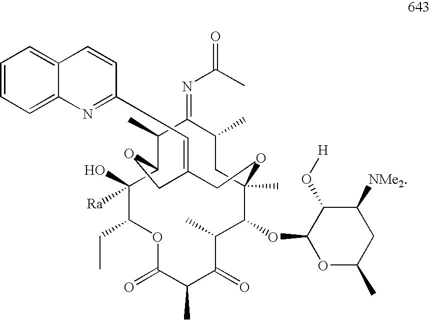

16. The compound of claim 1 having formula 580:

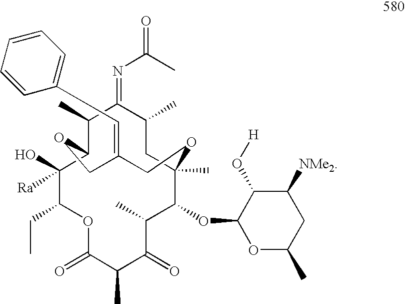

17. The compound of claim 1 having formula 958:

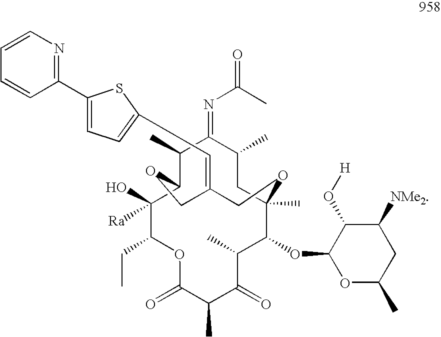

18. The compound of claim 1 having formula 015:

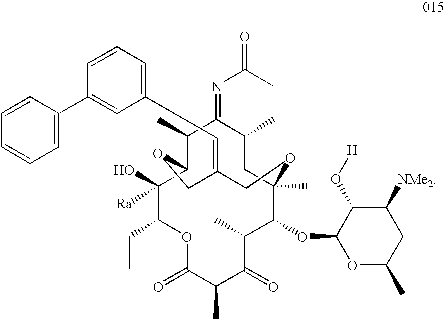

19. The compound of claim 1 having formula 174:

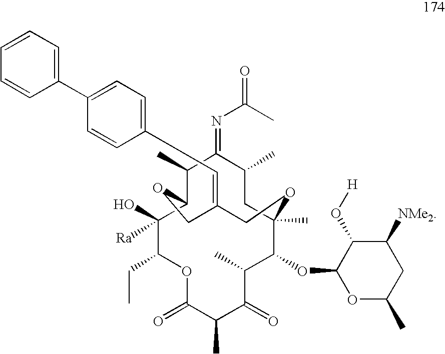

20. The compound of claim 1 having formula 264:

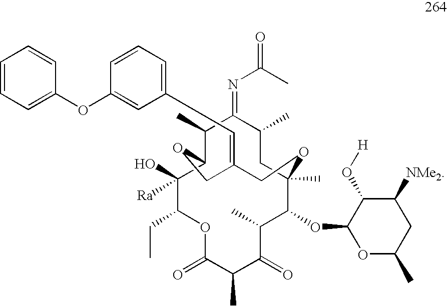

21. The compound of claim 1 having formula 413:

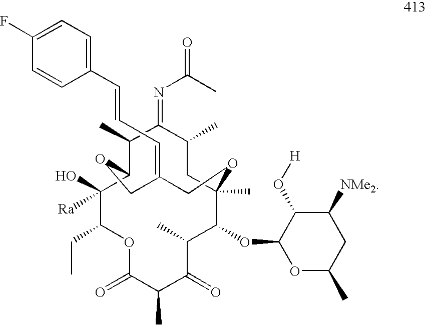

22. The compound of claim 1 having formula 598:

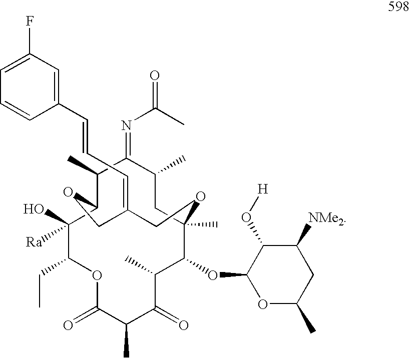

23. The compound of claim 1 having formula 873:

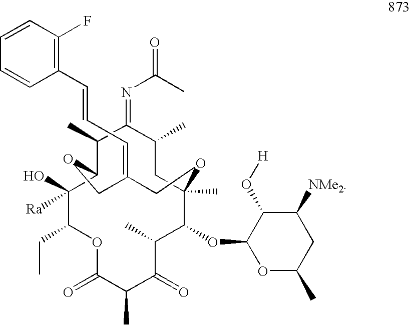

24. The compound of claim 1 having formula 253:

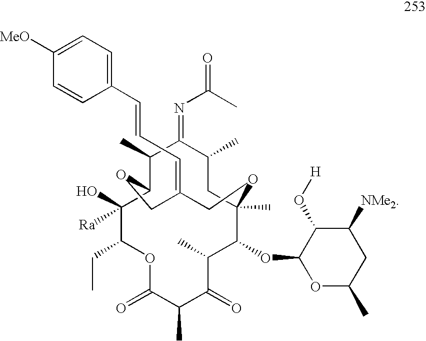

25. The compound of claim 1 having formula 271:

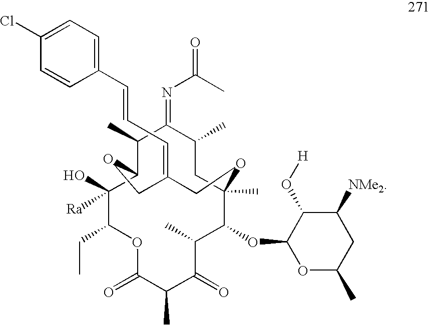

26. The compound of claim 1 having formula 202:

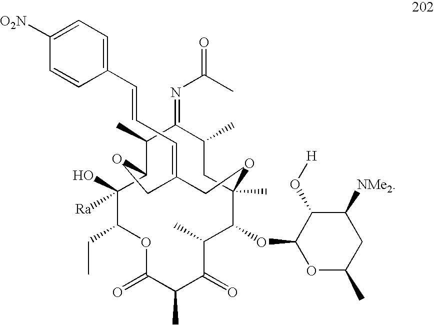

27. The compound of claim 1 having formula 952:

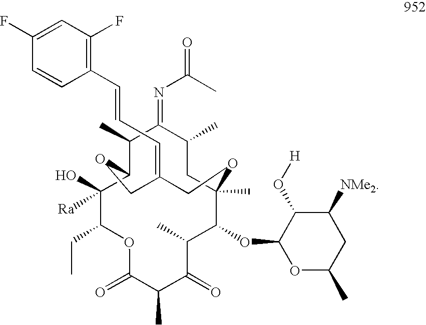

28. The compound of claim 1 having formula 608:

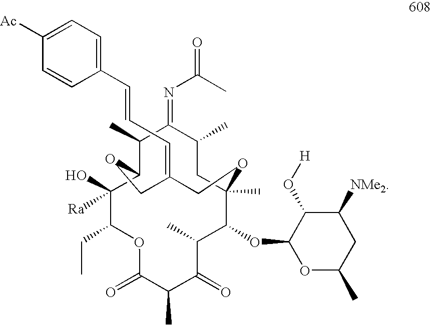

29. The compound of claim 1 having formula 668:

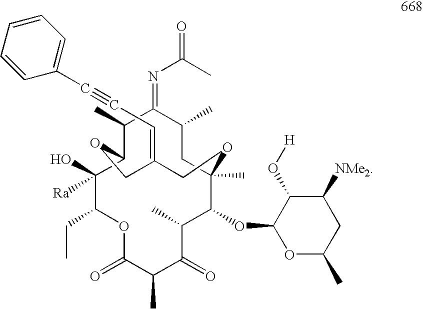

30. The compound of claim 1 having formula 926:

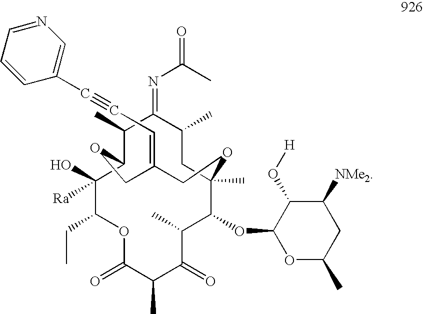

31. The compound of claim 1 having formula 026:

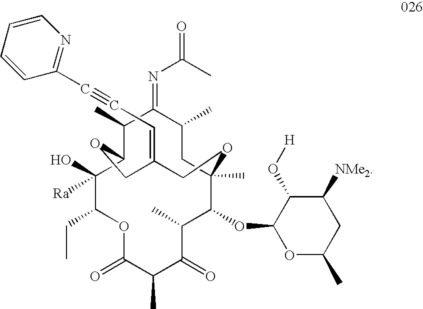

32. The compound of claim 1 having formula 687:

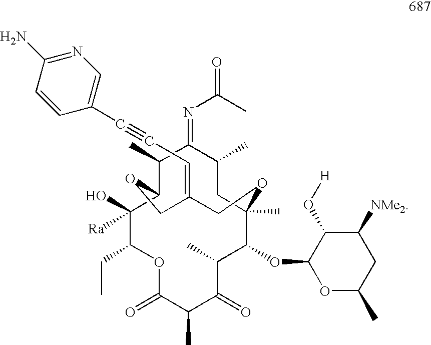

33. The compound of claim 1 having formula 592:

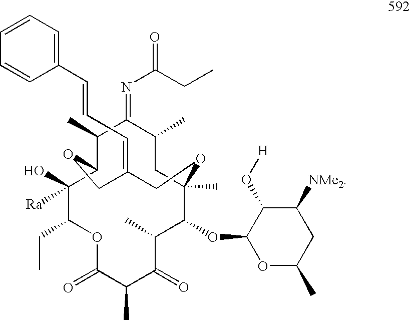

34. The compound of claim 1 having formula 941:

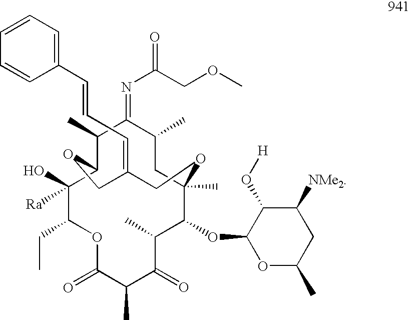

35. The compound of claim 1 having formula 676:

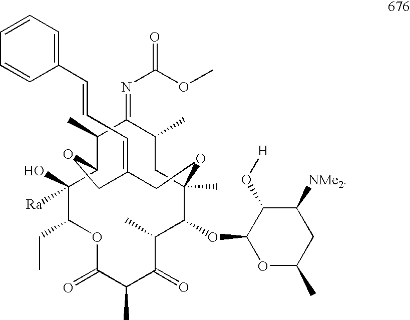

36. The compound of claim 1 having formula 367:

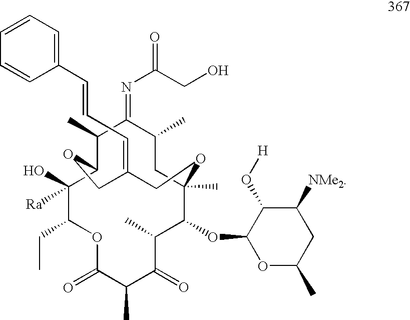

37. The compound of claim 1 having formula 771:

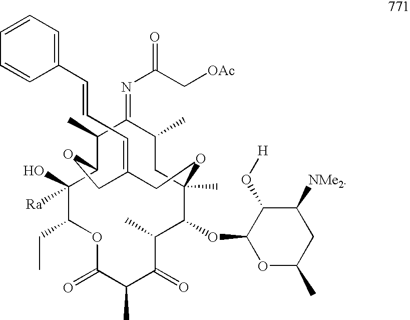

38. The compound of claim 1 having formula 052:

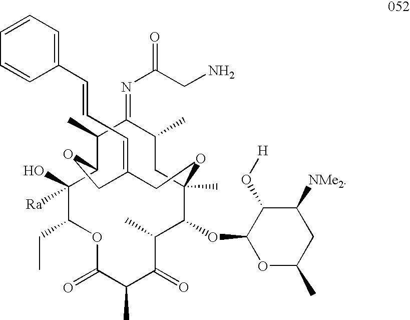

39. The compound of claim 1 having formula 668:

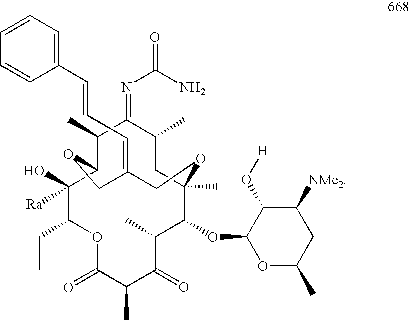

40. The compound of claim 1 having formula 932:

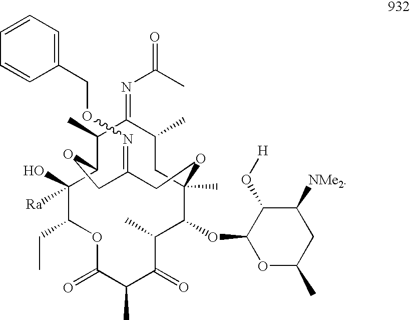

41. The compound of claim 1 having formula 230:

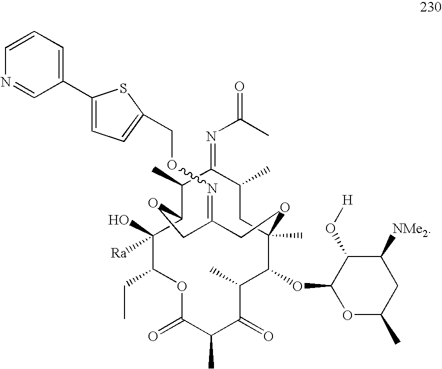

42. The compound of claim 1 having formula 911:

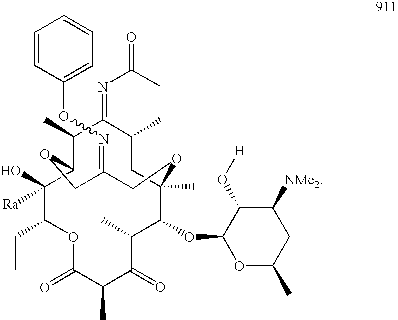

43. The compound of claim 1 having formula 841:

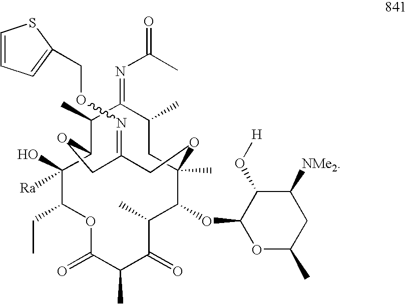

44. The compound of claim 1 having formula 629:

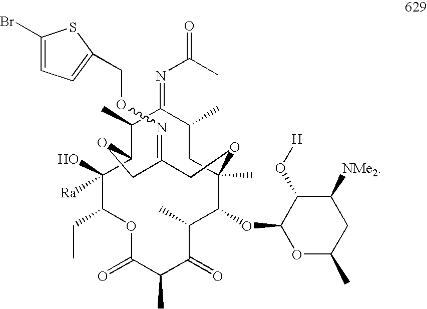

45. The compound of claim 1 having formula 512:

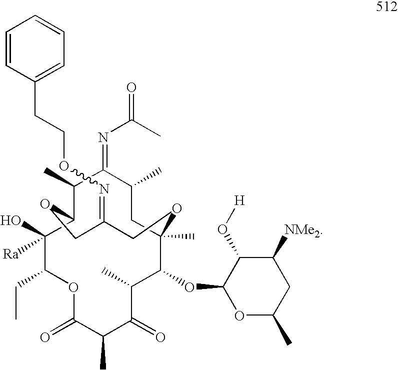

46. The compound of claim 1 having formula 914:

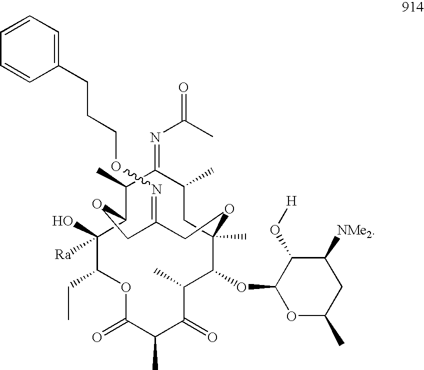

47. The compound of claim 1 having formula 664:

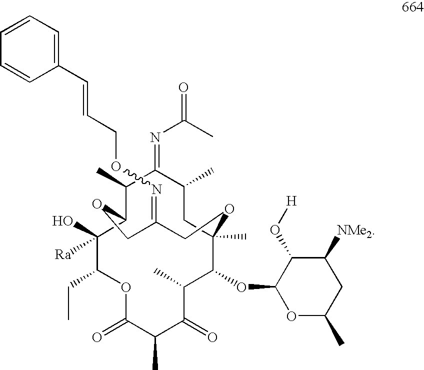

48. The compound of claim 1 having formula 889:

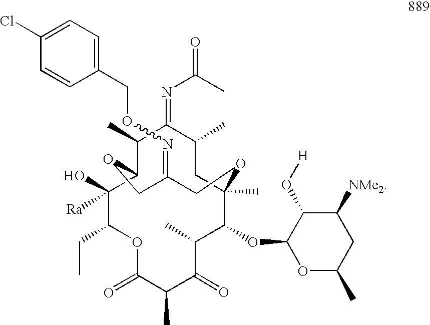

49. The compound of claim 1 having formula 568:

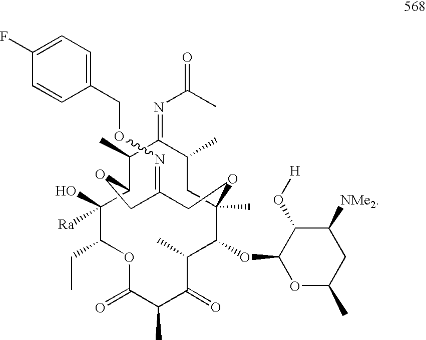

50. The compound of claim 1 having formula 753:

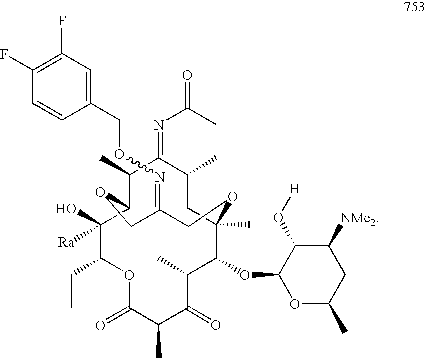

51. The compound of claim 1 having formula 459:

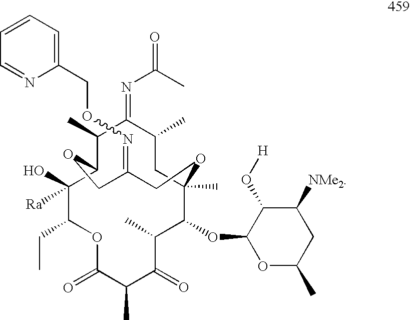

52. The compound of claim 1 having formula 220:

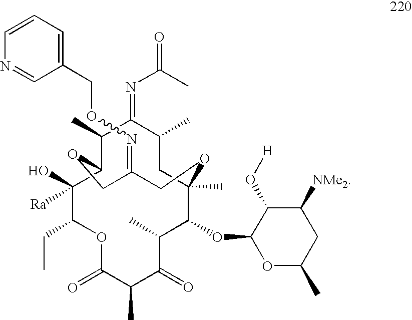

53. The compound of claim 1 having formula 311:

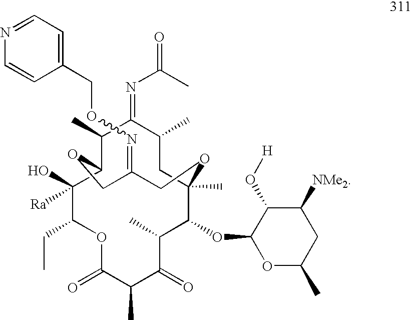

54. A pharmaceutical composition comprising a therapeutically effective amount of the compound of claim 1.

55. A method of treating bacterial infection in a patient in need thereof comprising administering to said patient a pharmaceutical composition comprising a therapeutically effective amount of the compound of claim 1.

56. The method of treating bacterial infection of claim 55, wherein said patient is a mammal.

57. The method of treating bacterial infection of claim 56, wherein said mammal is a human.

58. Use of a compound of any one of claims 1-53 in the manufacture of a medicament for the treatment or prophylaxis of bacterial infections.
